(19) United States
(12) Patent Application Publication
(10) Pub. No.: US 2020/0108074 A1
(43) Pub. Date: Apr. 9, 2020

(54) COMBINATIONS OF CHK1- AND WEE1- INHIBITORS

(71) Applicant: Seattle Genetics, Inc., Bothell, WA (US)

(72) Inventors: Alex VO, Bothell, WA (US); Kevin KLUCHER, Bothell, WA (US); Scott PETERSON, Bothell, WA (US)

(21) Appl. No.: 16/498,351

(22) PCT Filed: Mar. 30, 2018
(86) PCT No.: PCT/US2018/025464

§ 371 Date: Sep. 26, 2019

Related U.S. Application Data
(60) Provisional application No. 62/480,101, filed on Mar. 31, 2017.

(51) Int. Cl. A61K 31/522; A61P 35/04; A61K 45/06

(52) U.S. Cl. CPC A61K 31/522; A61K 45/06; A61P 35/04

(57) ABSTRACT

In one aspect, the present invention provides a method for preventing or treating cancer in a subject. In some embodiments, the method comprises administering a therapeutically effective amount of the Chk1 inhibitor Compound 1. In other embodiments, the method further comprises administering a therapeutically effective amount of a Wee1 inhibitor. Pharmaceutical compositions and kits are also provided herein.

### CROSS-REFERENCES TO RELATED APPLICATIONS

This application claims the benefit of U.S. Provisional Application No. 62/480,101 (filed Mar. 31, 2017). This application is incorporated by reference in its entirety for all purposes.

### FIELD OF THE INVENTION

The present invention is directed to compositions, methods, and uses related to the treatment of cancer. Various aspects and embodiments relate generally to Chk1 inhibitors (e.g., their combination with Wee1 inhibitors, and to methods of preparing or using such compounds and combinations in the treatment of cancer.

### BACKGROUND OF THE INVENTION

Cancer is a disease that imposes a substantial healthcare burden and significantly affects society in the United States and across the world. In the United States alone, it is estimated that over 1.6 million people were diagnosed with new cases of cancer in 2016, and that about 600,000 people died from cancer. Cancer is an extremely heterogeneous disease, with tumors arising from virtually every cell type in the body, and is associated with a wide range of environmental and genetic risk factors. Furthermore, cancer strikes people of all ages and of all ethnic, cultural, and socioeconomic groups.

Chk1 is a serine/threonine kinase that is involved in the induction of cell cycle checkpoints in response to DNA damage and replicative stress. Chk1 inhibition abrogates the intra S and G2/M checkpoints and has been shown to selectively sensitize tumor cells to well-known DNA damaging agents. (See, e.g., McNeely, S. et al. Pharmacology & Therapeutics 2014 (dx.doi.org/10.1016/j.pharmthera.2013.10.005)).

Resistance to chemotherapy and radiotherapy, a clinical problem for conventional therapy, has been associated with activation of the DNA damage response in which Chk1 has been implicated (Nature 2006; 444(7):756-760) and the inhibition of Chk1 sensitizes lung cancer brain metastases to radiotherapy (Biochem. Biophys. Res. Commun. 2011; 406(1):53-8).

Chk1 inhibitors, either as single agents or in combination, are useful, as an example, in treating tumor cells in which constitutive activation of DNA damage and checkpoint pathways drives genomic instability. Various attempts have been made to develop inhibitors of Chk1 kinase. For example, PCT Application Publication Nos. WO 2003/010444 and WO 2005/072733 disclose aryl/heteroaryl urea compounds as Chk1 kinase inhibitors. U.S. Patent Application Publication No. US 2005/0215556 discloses macrocyclic ureas as kinase inhibitors. PCT Application Publication Nos. WO 2002/070494, WO 2006/014359, and WO 2006/021002 disclose aryl and heteroaryl ureas as Chk1 inhibitors. PCT Application Publication Nos. WO 2011/141716 and WO 2013/072502 both disclose substituted pyrazinyl-phenyl ureas as Chk1 kinase inhibitors. PCT Application Publication Nos. WO 2005/009435 and WO 2010/077758 disclose aminopyrazoles as Chk1 kinase inhibitors.

Despite the aforementioned efforts, there remains a need for cell cycle checkpoint inhibitors that can be used as therapeutic agents to render cancer cells more susceptible to DNA damage and the activation of apoptosis pathways, and to make cancer cells less likely to become resistant to other chemotherapy and radiotherapy treatments. The present invention satisfies this need and provides related advantages as well.

### BRIEF SUMMARY OF THE INVENTION

In some aspects, the present disclosure provides a method for preventing or treating cancer in a subject, the method comprising administering to the subject a therapeutically effective amount of Compound 1 and a therapeutically effective amount of a Wee1 inhibitor. In some embodiments, the Wee1 inhibitor is adavosertib (i.e., AZD-1775).

In some embodiments, the cancer is selected from the group consisting of acute myeloid leukemia, esophageal cancer, gastric cancer, mantle cell lymphoma, non-small cell lung cancer (NSCLC), ovarian cancer, head and neck cancer, liver cancer, pancreatic cancer, prostate cancer, and a central nervous system cancer. In other embodiments, the cancer is a metastatic cancer. In some other embodiments, the cancer is a multidrug-resistant cancer.

In some embodiments, the dose of Compound 1 is between about 1 mg and 100 mg per kg of the subject's body weight. In some embodiments, the dose of Compound 1 is about 12.5 mg per kg of the subject's body weight. In other embodiments, the dose of Compound 1 is about 25 mg per kg of the subject's body weight. In some other embodiments, the dose of Compound 1 is about 50 mg per kg of the subject's body weight. In other embodiments, the dose of AZD-1775 is about 30 mg per kg of the subject's body weight. In particular embodiments, the dose of Compound 1 is about 25 mg per kg of the subject's body weight and the dose of AZD-1775 is about 30 mg per kg of the subject's body weight.

In some embodiments, Compound 1 and the Wee1 inhibitor are co-administered. In other embodiments, Compound 1 and the Wee1 inhibitor are co-administered simultaneously or sequentially. In particular embodiments, Compound 1 or the Wee1 inhibitor are administered orally, intravenously, intramuscularly, subcutaneously, or intratumorally.

In some embodiments, treating the subject results in a reduction of tumor volume. In other embodiments, treating the subject results in a decrease or elimination of one or more signs or symptoms of cancer. In some other embodiments, treating the subject results in an increased survival time. In particular embodiments, the administration is for prevention and the subject does not have cancer.

In other aspects, the present invention provides a pharmaceutical composition comprising Compound 1 and a pharmaceutically acceptable carrier. In some embodiments, the pharmaceutical composition further comprises a Wee1 inhibitor. In some embodiments, the Wee1 inhibitor is AZD-1775.

In some embodiments, Compound 1 is present at a concentration between about 0.1 nM and 2,000 nM. In some embodiments, the Wee1 inhibitor (e.g., AZD-1775) is present at a concentration between about 0.1 nM and 1,000 nM.

In yet other aspects, the present invention provides a kit for preventing or treating cancer in a subject, the kit comprising a pharmaceutical composition of the present invention. In some embodiments, the kit further comprises instructions for use. In some embodiments, the kit further comprises one or more reagents.

Other objects, features, and advantages of the present invention will be apparent to one of skill in the art from the following detailed description and figures.

### BRIEF DESCRIPTION OF THE DRAWINGS

FIG. 1 depicts the DNA damage response and cell cycle control axis in cancer.

FIG. 2 presents various drug properties of Compound 1.

FIGS. 3A-3D show that Compound 1 is a potent and selective inhibitor of Chk1. FIG. 3A depicts different kinase families. FIG. 3B shows the enzymatic selectivity of

Compound 1. “TBD” denotes that the value is to be determined. FIG. 3C shows the enzymatic potency of Compound 1. FIG. 3D shows the cellular potency of Compound 1.

FIG. 4 shows that Compound 1 was active in carcinoma cell lines that were derived from diverse histological origins. The horizontal axis intersects at the median IC₅₀ value for the total carcinoma population. IC₅₀ values are plotted relative to the median IC₅₀ for the carcinoma panel

FIGS. 5A-5D show that Compound 1 was active in non-small cell lung carcinoma (NSCLC) xenograft models. Data represent group mean+/−S.E.M. FIG. 5A depicts the effect of Compound 1 on tumor volume in an SK-MES NSCLC tumor model. FIG. 5B depicts the effect of Compound 1 on tumor volume in an NCI-H727 NSCLC tumor model. FIG. 5C depicts the effect of Compound 1 on body weight in an SK-MES NSCLC tumor model. FIG. 5D depicts the effect of Compound 1 on body weight in an NCI-H727 NSCLC tumor model.

FIGS. 6A-C show that Compound 1 and AZD-1775 were synergistic and exhibited unique patterns of cellular activity as single agents. Data represent group means+/−S.E.M. Black arrows represent the signal corresponding to the starting cell number (cytostatic limit). FIG. 6A depicts the effects of various combinations of Compound 1 and AZD-1775 on cell viability in an SK-MES NSCLC tumor model. FIG. 6B depicts the effects of various combinations of Compound 1 and AZD-1775 on cell viability in an NCI-H727 NSCLC tumor model. FIG. 6C shows a comparison of Compound 1 and AZD-1775, depicting IC₅₀ values in various cancer cell lines.

FIGS. 7A and 7B show that Compound 1 and AZD-1775 were active in an NCI-H727 NSCLC xenograft tumor model. Data represent group means+/−S.E.M. FIG. 7A depicts the effects of Compound 1 and AZD-1775, alone and in combination, on tumor volume. FIG. 7B depicts the effects of Compound 1 and AZD-1775, alone and in combination, on body weight.

FIG. 8 shows the results of a screen of hematopoietic cell lines for sensitivity to Compound 1.

FIGS. 9A and 9B show that Compound 1 demonstrated single-agent anti-proliferative activity in mantle cell lymphoma cell lines. FIG. 9A shows the results of a proliferation assay. FIG. 9B shows IC₅₀ values for the cell lines depicted in FIG. 9A.

FIGS. 10A-10D show that Compound 1 was active and well-tolerated as a single agent in mantle cell lymphoma tumor xenograft models. FIG. 10A shows the effects of Compound 1 on Jeko-1 tumor growth. FIG. 10B shows the effects of Compound 1 on body weight in the Jeko-1 tumor model. FIG. 10C shows the effects of Compound 1 on Maver-1 tumor growth. FIG. 10D shows the effects of Compound 1 on body weight in the Maver-1 tumor model.

FIGS. 11A-11D show that Compound 1 in combination with a Wee1 inhibitor showed synergistic anti-proliferative effects in mantle cell lymphoma cell lines. FIG. 11A shows the effects of Compound 1 and AZD-1775 on Jeko-1 cells. FIG. 11B shows the effects of Compound 1 and AZD-1775 on Maver-1 cells. FIG. 11C shows the effects of Compound 1 and AZD-1775 on Z-138 cells. FIG. 11D shows combination indices for Jeko-1, Z-138, and Maver-1 cells.

FIG. 12 shows the results of phospho-H2A.X assays in Jeko-1, Z-138, and Maver-1 cells.

FIGS. 13A-13C show that Compound 1 induction of apoptosis in mantle cell lymphoma cell lines was increased with concurrent Wee1 inhibition. FIG. 13A shows the results of a caspase-3/7 assay in Jeko-1 cells. FIG. 13B shows the results of a caspase-3/7 assay in Maver-1 cells. FIG. 13C shows the results of a caspase-3/7 assay in Z-138 cells.

FIGS. 14A and 14B show that the anti-tumor activity of Compound 1 was enhanced when combined with a Wee1 inhibitor in a Jeko-1 mantle cell lymphoma tumor model. FIG. 14A shows the effects of Compound 1 and AZD-1775 on tumor growth. FIG. 14B shows the effects of Compound 1 and AZD-1775 on body weight.

FIGS. 15A-15C show that Compound 1 demonstrated anti-proliferative activity and induced DNA damage in AML cell lines. FIG. 15A shows the results of a proliferation assay in multiple cell lines. FIG. 15B shows the Compound 1 IC₅₀ values in the cell lines depicted in FIG. 15A. FIG. 15C shows the results of a phospho-H2A.X (S139) assay.

FIGS. 16A and 16B show that Compound 1 was active and well-tolerated as a single agent in an MV-411 AML tumor xenograft model. FIG. 16A shows the effects of Compound 1 on MV-411 tumor growth. FIG. 16B shows the effects of Compound 1 on body weight in the MV-411 tumor model.

### DETAILED DESCRIPTION OF THE INVENTION

### I. Introduction

Checkpoint kinase 1 (Chk1) is a serine/threonine protein kinase that regulates cell division in response to genotoxic stress by arresting cell cycle progression in the S & G2 phases. Pharmacological inhibition of Chk1 targets tumor cells with increased DNA replication stress, resulting in the uncoupling of DNA replication checkpoint function and the induction of DNA damage and cell death. These properties make Chk1 inhibition a novel therapeutic approach as a single agent in cancers with high replication stress that is driven by oncogenic signaling and loss of parallel DNA damage response pathway function.

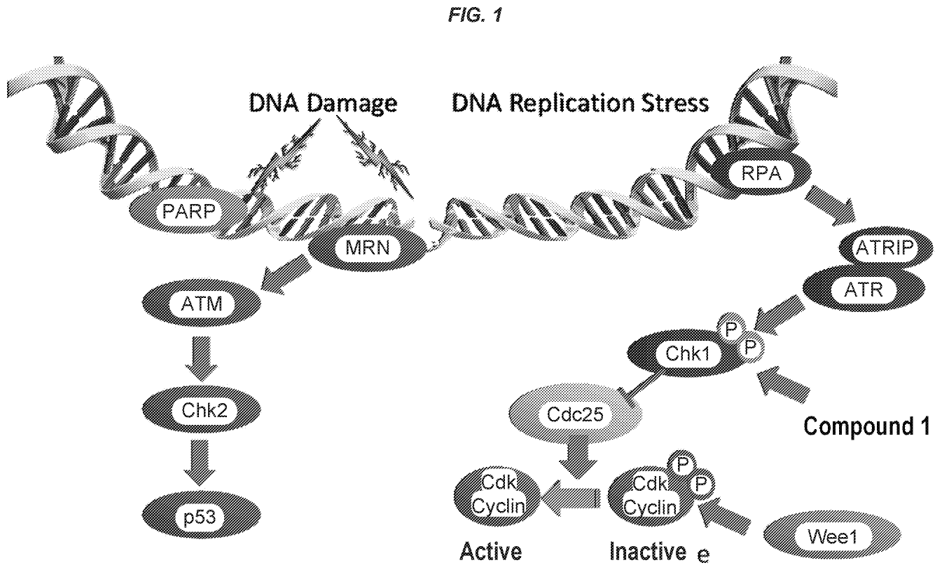

Targeting cell cycle regulation and DNA damage response (DDR) signaling is a clinically validated approach to cancer therapy. As shown in FIG. 1, Chk1 is a key modulator of the cell division cycle, as well as cellular DDR signaling. Chk1 regulates the cell division cycle in response to DNA damage and DNA replication stress. Furthermore, Chk1 functions in parallel with other DDR and cell cycle regulatory pathways, many of which are deregulated in cancer cells. Loss of DDR and cell cycle regulation in cancers increases sensitivity to Chk1 inhibition.

Cell division cycle 25 (Cdc25) is a phosphatase that activates cyclins and results in increased cyclin-dependent kinase (Cdk) activity. Chk1 inhibitors block cell cycle checkpoint activation by disrupting the control of Cdc25 by Chk1, resulting in increased cyclin and Cdk activity. In addition, as shown in FIG. 1, Wee1 functions in parallel with Chk1 to regulate cyclin activation and Cdk activity.

The present invention is based, in part, on the discovery that the Chk1 inhibitor Compound 1 inhibits tumor growth and cell viability in a wide range of cancer cell lines that correspond to a number of different cancers. The present invention is also based, in part, on the discovery that Compound 1 exhibits a synergistic effect when given in combination with an inhibitor of Wee1 , another protein that functions as a cell cycle checkpoint regulator. The combination treatment of Compound 1 and an inhibitor of Wee1 results in a Chou-Talalay combination index (CI) of less than 1. Chou-Talalay is a widely used method that offers a quantitative definition for additive effect (CI=1), synergism (CI<1), and antagonism (CI>1) in drug combinations. See, e.g., Chou, T. C., Cancer Res. 2010, 70(2), 440-6.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11 mg/kg” is equivalent to “about 7, about 9, or about 11 mg/kg.”

The term “or” as used herein should in general be construed non-exclusively. For example, a claim to “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

The group “A or B” is typically equivalent to the group “selected from the group consisting of A and B.”

The term “comprising” as used herein should in general be construed as not excluding additional ingredients. For example, a claim to “a composition comprising A” would cover compositions that include A and B; A, B, and C; A, B, C, and D; A, B, C, D, and E; and the like.

The terms “subject,” “individual,” and “patient” as used herein are used interchangeably herein to refer to a vertebrate, preferably a mammal, more preferably a human. Mammals include, but are not limited to, murines, rats, simians, humans, farm animals, sport animals, and pets. Tissues, cells and their progeny of a biological entity obtained in vivo or cultured in vitro are also encompassed.

As used herein, the term “therapeutically effective amount” includes a dosage sufficient to produce a desired result with respect to the indicated disorder, condition, or mental state. The desired result may comprise a subjective or objective improvement in the recipient of the dosage. For example, an effective amount of a Chk1 inhibitor, such as Compound 1; a Wee1 inhibitor, such as AZD-1775; or a combination of a Chk1 inhibitor and a Wee1 inhibitor includes an amount sufficient to alleviate the signs, symptoms, or causes of cancer (e.g., acute myeloid leukemia, esophageal cancer, gastric cancer, mantle cell lymphoma, non-small cell lung cancer (NSCLC), ovarian cancer, head and neck cancer, liver cancer, pancreatic cancer, prostate cancer, or a central nervous system cancer). As another example, an effective amount of a Chk1 inhibitor, such as Compound 1; a Wee1 inhibitor, such as AZD-1775; or a combination of a Chk1 inhibitor and a Wee1 inhibitor includes an amount sufficient to alleviate the signs, symptoms, or causes of metastatic or multidrug-resistant cancer. As another example, an effective amount of a Chk1 inhibitor, such as Compound 1; a Wee1 inhibitor, such as AZD-1775; or a combination of a Chk1 inhibitor and a Wee1 inhibitor includes an amount sufficient to prevent the development of a cancer.

Thus, a therapeutically effective amount can be an amount that slows, reverses, or prevents tumor growth, increases survival time, or inhibits tumor progression or metastasis. Also, for example, an effective amount of a Chk1 inhibitor, such as Compound 1; a Wee1 inhibitor, such as AZD-1775; or a combination of a Chk1 inhibitor and a Wee1 inhibitor includes an amount sufficient to cause a substantial improvement in a subject having cancer when administered to the subject. The effective mount can vary with the type and stage of the cancer being treated, the type and concentration of one or more compositions (e.g., comprising a Chk1 inhibitor, such as Compound 1; a Wee1 inhibitor, such as AZD-1775; or a combination of a Chk1 inhibitor and a Wee1 inhibitor) administered, and the amounts of other drugs that are also administered.

For the purposes herein, a therapeutically effective amount is determined by such considerations as may be known in the art. The amount must be effective to achieve the desired therapeutic effect in a subject suffering from cancer. The therapeutically effective amount depends, inter alia, on the type and severity of the disease to be treated and the treatment regimen. The therapeutically effective amount is typically determined in appropriately designed clinical trials (e.g., dose range studies) and the person versed in the art will know how to properly conduct such trials in order to determine the therapeutically effective amount. As generally known, a therapeutically effective amount depends on a variety of factors including the distribution profile of a therapeutic agent (e.g., a Chk1 inhibitor, such as Compound 1; a Wee1 inhibitor, such as AZD-1775; or a combination of a Chk1 inhibitor and a Wee1 inhibitor) or composition within the body, the relationship between a variety of pharmacological parameters (e.g., half-life in the body) and undesired side effects, and other factors such as age and sex, etc.

The term “survival” or “survival time” refers to a length of time following the diagnosis of a disease or beginning or completing a particular course of therapy for a disease (e.g., cancer). The term “overall survival” includes the clinical endpoint describing patients who are alive for a defined period of time after being diagnosed with or treated for a disease, such as cancer. The term “disease-free survival” includes the length of time after treatment for a specific disease (e.g., cancer) during which a patient survives with no sign of the disease (e.g., without known recurrence). In certain embodiments, disease-free survival is a clinical parameter used to evaluate the efficacy of a particular therapy, which is usually measured in units of 1 or 5 years. The term “progression-free survival” includes the length of time during and after treatment for a specific disease (e.g., cancer) in which a patient is living with the disease without additional symptoms of the disease. In some embodiments, survival is expressed as a median or mean value.

As used herein, the term “treating” includes, but is not limited to, methods and manipulations to produce beneficial changes in a recipient's health status (e.g., a patient's cancer status). The changes can be either subjective or objective and can relate to features such as symptoms or signs of the cancer being treated. For example, if the patient notes decreased pain, then successful treatment of pain has occurred. For example, if a decrease in the amount of swelling has occurred, then a beneficial treatment of inflammation has occurred. Similarly, if the clinician notes objective changes, such as reducing the number of cancer cells, the growth of the cancer cells, the size of cancer tumors, or the resistance of the cancer cells to another cancer drug, then treatment of cancer has also been beneficial. Preventing the deterioration of a recipient's status is also included by the term. Treating, as used herein, also includes administering a Chk1 inhibitor, such as Compound 1, and a Wee1 inhibitor, such as AZD-1775; or a combination of a Chk1 inhibitor and a Wee1 inhibitor to a patient having cancer (e.g., acute myeloid leukemia, esophageal cancer, gastric cancer, mantle cell lymphoma, non-small cell lung cancer (NSCLC), ovarian cancer, head and neck cancer, liver cancer, pancreatic cancer, prostate cancer, or a central nervous system cancer).

The terms “administering” and “administration” include oral administration, topical contact, administration as a suppository, intravenous, intraperitoneal, intramuscular, intralesional, intratumoral, intrathecal, intranasal (e.g., inhalation, nasal mist or drops), or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. One skilled in the art will know of additional methods for administering a therapeutically effective amount of a Chk1 inhibitor, such as Compound 1; a Wee1 inhibitor, such as AZD-1775; or a combination of a Chk1 inhibitor and a Wee1 inhibitor according to methods of the present invention for preventing or relieving one or more symptoms associated with cancer.

As used herein, the term “co-administering” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. The two or more structurally different compounds can be comprised of a Chk1 inhibitor (e.g., Compound 1) and a Wee1 inhibitor (e.g., AZD-1775). In some embodiments, the co-administered compounds are administered by the same route. In other embodiments, the co-administered compounds are administered via different routes. For example, one compound can be administered orally, and the other compound can be administered, e.g., sequentially or simultaneously, via intravenous, intramuscular, subcutaneous, or intraperitoneal injection. The simultaneously or sequentially administered compounds or compositions can be administered such that a Chk1 inhibitor and a Wee1 inhibitor are simultaneously present in a subject or in a cell at an effective concentration.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the invention and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some embodiments, the carrier is an agent that facilitates the delivery of a peptide or an oligonucleotide to a target cell or tissue. One of skill in the art will recognize that other pharmaceutical carriers are useful in the present invention.

As used herein, the term “cancer” is intended to include a member of a class of diseases characterized by the uncontrolled growth of aberrant cells. The term includes cancers of all stages and grades including advanced, recurrent, pre- and post-metastatic cancers. Drug-resistant and multidrug-resistant cancers are also included. Cancers suitable for treatment according to methods of the present invention include gastric cancer, lung cancers (e.g., non-small cell lung cancer (NSCLC)), ovarian cancer, breast cancer, colorectal cancer, nervous system cancers (e.g., central nervous system cancers), adrenal gland cancer, bladder cancer, blood cancers (e.g., leukemia, acute myeloid leukemia, mantle cell lymphoma, anaplastic large cell lymphoma (ALCL), B-cell acute lymphoblastic leukemia, Burkitt lymphoma, chronic lymphocytic leukemia, chronic myelogenous leukemia, multiple myeloma, acute promyelocytic leukemia, T-cell acute lymphoblastic leukemia), bone cancer, cervical cancer, esophageal cancer, eye cancer, renal cancer, head and neck cancer, liver cancer, muscle cancer, nasal cancer, pancreatic cancer, pharyngeal cancer, placental cancer, prostate cancer, skin cancer, soft tissue cancers, submaxillary gland cancer, thyroid cancer, tongue cancer, and uterine cancer. As used herein, a “tumor” comprises one or more cancerous cells. Combinations of cancer are not excluded by the term.

In the context of cancer, the term “stage” refers to a classification of the extent of cancer. Factors that are considered when staging a cancer include but are not limited to tumor size, tumor invasion of nearby tissues, and whether the tumor has metastasized to other sites. The specific criteria and parameters for differentiating one stage from another can vary depending on the type of cancer. Cancer staging is used, for example, to assist in determining a prognosis or identifying the most appropriate treatment option(s).

One non-limiting example of a cancer staging system is referred to as the “TNM” system. In the TNM system, “T” refers to the size and extent of the main tumor, “N” refers to the number of nearby lymph nodes to which the cancer has spread, and “M” refers to whether the cancer has metastasized. “TX” denotes that the main tumor cannot be measured, “T0” denotes that the main tumor cannot be found, and “T1,” “T2,” “T3,” and “T4” denote the size or extent of the main tumor, wherein a larger number corresponds to a larger tumor or a tumor that has grown into nearby tissues. “NX” denotes that cancer in nearby lymph nodes cannot be measured, “N0” denotes that there is no cancer in nearby lymph nodes, and “N1,” “N2,” “N3,” and “N4” denote the number and location of lymph nodes to which the cancer has spread, wherein a larger number corresponds to a greater number of lymph nodes containing the cancer. “MX” denotes that metastasis cannot be measured, “M0” denotes that no metastasis has occurred, and “M1” denotes that the cancer has metastasized to other parts of the body.

As another non-limiting example of a cancer staging system, cancers are classified or graded as having one of five stages: “Stage 0,” “Stage I,” “Stage II,” “Stage III,” or “Stage IV.” Stage 0 denotes that abnormal cells are present, but have not spread to nearby tissue. This is also commonly called carcinoma in situ (CIS). CIS is not cancer, but may subsequently develop into cancer. Stages I, II, and III denote that cancer is present. Higher numbers correspond to larger tumor sizes or tumors that have spread to nearby tissues. Stage IV denotes that the cancer has metastasized. One of skill in the art will be familiar with the different cancer staging systems and readily be able to apply or interpret them.

The term “Compound 1” refers to 5-((5-(4-(4-fluoro-1-methylpiperidin-4-yl)-2-methoxyphenyl)-1H-pyrazol-3-yl)amino)pyrazine-2-carbonitrile, which acts as an inhibitor of Chk1.

The term “AZD-1775,” also known as “AZD1775,” “MK-1775,” and “MK1775,” refers to 2-allyl-1-(6-(2-hydroxypropan-2-yl)pyridin-2-yl)-6-((4-(4-methylpiperazin-1-yl)phenyl)amino)-1H-pyrazolo[3,4-d]pyrimidin-3(2H)-one, which has the following structure:

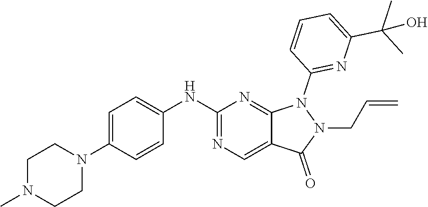 [SMILES: C=CCn1c(=O)c2cnc(Nc3ccc(N4CCN(C)CC4)cc3)nc2n1-c1cccc(C(C)(C)O)n1]

AZD-1775 is a highly selective, ATP competitive, small molecule inhibitor of Wee1 , having an enzyme IC₅₀ of about 5.18 nM. In vitro, AZD-1775 inhibits Wee1 activity and induces DNA damage as well as G2 checkpoint escape in cell-based assays with an EC₅₀ of about 80 nM. AZD-1775 increases cytotoxicity when used in combination with DNA damaging agents, such as gemcitabine, cisplatin, carboplatin and topotecan, in p53-deficient cell lines.

The term “checkpoint kinase 1” or “Chk1” refers to the serine/threonine kinase also known as “CHEK1” that is encoded by the CHEK1 gene in humans. Chk1 coordinates the DNA damage response (DDR) and the cell cycle checkpoint response. Chk1 activation results in cell cycle checkpoint activation, cell cycle arrest, DNA repair, and cell death. Chk1 is activated in response to phosphorylation by ATR, which can be triggered by the detection of single strand DNA that can result from UV-induced damage, DNA replication stress, and inter-strand crosslinking. Other proteins such as replication protein A, Claspin, the Tim-Tipin complex, Rad 17, and DNA topoisomerase 2-binding protein 1 (TopBP1) are involved in Chk1 activation. In addition, proteins such as the kinases PKB/AKT, MAPKAPK, and p90/RSK are involved in ART-independent activation of Chk1.

One of the primary targets of Chk1 is the phosphatase Cdc25, which is inhibited by Chk1, resulting in inactivation of cyclins and cyclin-dependent kinase (Cdk) activity, which are key drivers of the cell cycle. Thus, Chk1 inhibition promotes activation of cyclins and Cdk activity, and ultimately progression through the cell cycle.

Non-limiting examples of human Chk1 mRNA sequences are set forth under GenBank reference numbers NM_001114121→NP_001107593, NM_001114122→NP_001107594, NM_001244846→NP_001231775, NM_001274→NP_001265, and NM_001330427→NP_001317356.

The term “Chk1 inhibitor” refers to any compound (e.g., a pharmaceutically active compound) that reduces or eliminates Chk1 activity. Chk1 inhibitors, for example, can result in the reduction or elimination of Chk1 activation by one or more signaling molecules, proteins, or other compounds (e.g., a Chk1 inhibitor can decrease or eliminate Chk1 activation in response to phosphorylation by ATR), or can result in the reduction or elimination of Chk1 activation by all signaling molecules, proteins, or other compounds. The term also includes compounds that decrease or eliminate the activation or deactivation of one or more proteins or cell signaling components by Chk1 (e.g., a Chk1 inhibitor can decrease or eliminate Chk1-dependent inhibition of Cdc25 phosphatase activity). Chk1 inhibitors also include compounds that inhibit Chk1 expression (e.g., compounds that inhibit Chk1 transcription or translation).

The term “Wee1” refers to a nuclear serine/threonine kinase encoded by the WEE1 gene in humans. Wee1 is also known as “Wee1 G2 checkpoint kinase” and “Wee1A kinase”. Wee1 activates cell cycle checkpoints by phosphorylating and thus inhibiting cyclin and Cdk activity. Wee1 functions in regulation of the G2/M checkpoint, the cell size checkpoint, and the DNA damage checkpoint. In higher eukaryotes, Wee1 is inactivated by phosphorylation and degradation. The SCF protein complex (an E3 ubiquitin ligase) regulates Wee1 by ubiquitination. Additionally, recognition of Wee1 by SCF is mediated by phosphorylation of Wee1 by Polio-like kinase 1 (Plk1) and Cdc2. Wee1 is also negatively regulated by Kruppel-like factor 2 (Klf2). A non-limiting example of a human Wee1 mRNA sequence is set forth under GenBank reference number NM_003390→NP_003381.

The term “Wee1 inhibitor” refers to any compound (e.g., a pharmaceutically active compound) that reduces or eliminates Wee1 activity. Wee1 inhibitors, for example, can result in the reduction or elimination of Wee1 activation by one or more signaling molecules, proteins, or other compounds, or can result in the reduction or elimination of Wee1 activation by all signaling molecules, proteins, or other compounds. The term also includes compounds that decrease or eliminate the activation or deactivation of one or more proteins or cell signaling components by Wee1 (e.g., a Wee1 inhibitor can decrease or eliminate Wee1-dependent inactivation of cyclin and Cdk activity). Wee1 inhibitors also include compounds that inhibit Wee1 expression (e.g., compounds that inhibit Wee1 transcription or translation).

Besides AZD-1775, other examples of Wee1 inhibitors are described in, e.g., U.S. Pat. Nos. 7,834,019; 7,935,708; 8,288,396; 8,436,004; 8,710,065; 8,716,297; 8,791,125; 8,796,289; 9,051,327; 9,181,239; 9,714,244; 9,718,821; and 9,850,247; U.S. Pat. App. Pub. Nos. US 2010/0113445 and 2016/0222459; and Intl Pat. App. Pub. Nos. WO 2002/090360, 2015/019037, 2017/013436, 2017/216559, 2018/011569, and 2018/011570. The disclosures of these patents and publications are incorporated herein by reference.

### III. Description of the Embodiments

### A. Methods for Preventing and Treating Cancer

In one aspect, the present disclosure provides a method for preventing or treating cancer (e.g., acute myeloid leukemia, esophageal cancer, gastric cancer, mantle cell lymphoma, non-small cell lung cancer (NSCLC), ovarian cancer, head and neck cancer, liver cancer, pancreatic cancer, prostate cancer, or a central nervous system cancer) in a subject, the method comprising administering to the subject a therapeutically effective amount of a checkpoint kinase 1 (Chk1) inhibitor.

Chk1 inhibitors suitable for the prevention or treatment of cancer according to methods of the present invention are disclosed in PCT Application Publication No. WO 2015/120390, hereby incorporated by reference in its entirety for all purposes. In particular embodiments, the Chk1 inhibitor is Compound 1 or a pharmaceutically acceptable salt thereof.

In some embodiments, the method further comprises administering to the subject a therapeutically effective amount of a Wee1 inhibitor. In some embodiments, the Wee1 inhibitor is selected from the group consisting of a pyrazolopyrimidine derivative, a pyridopyrimidine, 4-(2-chlorophenyl)-9-hydroxypyrrolo[3,4-c]carbazole-1,3-(2H, 6H)-dione (also known as “Wee1 Inhibitor” or “Chk1 Inhibitor V” (CAS No. 622855-37-2)), 6-butyl-4-(2-chlorophenyl)-9-hydroxypyrrolo[3,4-c]carbazole-1,3-(2H,6H)-dione (also known as “Wee1 Inhibitor II” (CAS No. 62285550-9)), 4-(2-phenyl)-9-hydroxypyrrolo[3,4-c]carbazole-1,3-(2H,6H)-dione (also known as “Wee1 Inhibitor III” or “Chk1 Inhibitor IV” (CAS No. 1177150-89-8)), an anti-Wee1 antibody, and an anti-Wee1 small interfering RNA (siRNA) molecule. In some embodiments, the pyridopyrimidine is pyrido [2,3-d] pyrimidine (also known as PD0166285 or 6-(2,6-dichlorophenyl)-2-[4-[2-(diethylamino)ethoxy]anilino]-8-methylpyrido[2,3-d]pyrimidin-7-one). In some embodiments, the pyrazolopyrimidine derivative is AZD-1775. In some embodiments, the pyrazolopyrimidine is methyl 4-(4-((2-allyl-1-(6-(2-hydroxypropan-2-yl)pyridin-2-yl)-3 -oxo-2,3 -dihydro-1H-pyrazolo[3,4-d]pyrimidin-6-yl)amino)phenyl)piperazine-1-carboxylate (i.e., CJM061). In some embodiments, the pyrazolopyrimidine is an analogous compound with a different carbamate group (e.g., ethyl).

Methods of the present disclosure are suitable for preventing or treating any number of cancers. In some embodiments, the type of cancer that is prevented or treated is selected from the group consisting of gastric cancer, a lung cancer (e.g., non-small cell lung cancer (NSCLC)), ovarian cancer, breast cancer, colorectal cancer, head and neck cancer, a nervous system cancer (e.g., central nervous system cancers), adrenal gland cancer, bladder cancer, a blood cancer (e.g., leukemia, acute myeloid leukemia, mantle cell lymphoma, anaplastic large cell lymphoma, B-cell acute lymphoblastic leukemia, Burkitt lymphoma, chronic lymphocytic leukemia, chronic myelogenous leukemia, multiple myeloma, acute promyelocytic leukemia, T-cell acute lymphoblastic leukemia), bone cancer, cervical cancer, esophageal cancer, eye cancer, renal cancer, liver cancer, muscle cancer, nasal cancer, pancreatic cancer, pharyngeal cancer, placental cancer, prostate cancer, skin cancer, soft tissue cancers, submaxillary gland cancer, thyroid cancer, tongue cancer, and uterine cancer. In particular embodiments, the cancer that is prevented or treated is selected from the group consisting of acute myeloid leukemia, gastric cancer, esophageal cancer, mantle cell lymphoma, non-small cell lung cancer (NSCLC), ovarian cancer, head and neck cancer, liver cancer, pancreatic cancer, prostate cancer, and a central nervous system cancer. In some embodiments, the cancer is a metastatic cancer. In some embodiments, the cancer is an advanced cancer. In some embodiments, the cancer is a drug-resistant cancer. In some embodiments, the cancer is a multidrug-resistant cancer. In some embodiments, the cancer is advanced, metastatic, or drug-resistant. In some embodiments, the cancer is mantle cell lymphoma. In some embodiments, the cancer is mantle cell lymphoma and the subject has a chromosomal translocation at t(11;14)(q13;q32). In particular embodiments, the cancer is a breast cancer or metastatic breast cancer.

In some embodiments, the therapeutically effective amount of the Chk1 inhibitor (e.g., Compound 1) is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In other embodiments, the therapeutically effective amount of the Chk1 inhibitor (e.g., Compound 1) is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, the therapeutically effective amount of the Chk1 inhibitor (e.g., Compound 1) is at least about 100 mg to 500 mg per kg of the subject's body weight (e.g., at least about 100, 125, 150, 175, 200, 225, 250, 275, 300, 325, 350, 375, 400, 425, 450, 475, or 500 mg per kg of the subject's body weight). In some embodiments, the therapeutically effective amount of the Chk1 inhibitor (e.g., Compound 1) is between about 1 mg and 50 mg per kg of the subject's body weight (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg per kg of the subject's body weight). In some embodiments, the therapeutically effective amount of the Chk1 inhibitor (e.g., Compound 1) is about 7.5 mg per kg of the subject's body weight. In some embodiments, the therapeutically effective amount of the Chk1 inhibitor (e.g., Compound 1) is about 12.5 mg per kg of the subject's body weight. In some other embodiments, the therapeutically effective amount of the Chk1 inhibitor (e.g., Compound 1) is about 20 mg per kg of the subject's body weight. In other embodiments, the therapeutically effective amount of the Chk1 inhibitor (e.g., Compound 1) is about 25 mg per kg of the subject's body weight. In some embodiments, the therapeutically effective amount of the Chk1 inhibitor (e.g., Compound 1) is about 50 mg per kg of the subject's body weight.

In some embodiments, a dose of the Chk1 inhibitor (e.g., Compound 1) comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of the Chk1 inhibitor. In other embodiments, a dose of the Chk1 inhibitor (e.g., Compound 1) comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of the Chk1 inhibitor.

In some embodiments, a dose of the Chk1 inhibitor (e.g., Compound 1) is at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of the Chk1 inhibitor.

In some embodiments, a dose of the Chk1 inhibitor (e.g., Compound 1) contains a therapeutically effective amount of the Chk1 inhibitor. In other embodiments, a dose of the Chk1 inhibitor (e.g., Compound 1) contains less than a therapeutically effective amount of the Chk1 inhibitor if administered without a Wee1 inhibitor.

In some embodiments, the therapeutically effective amount of the Wee1 inhibitor (e.g., AZD-1775) is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In other embodiments, the therapeutically effective amount of the Wee1 inhibitor (e.g., AZD-1775) is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, the therapeutically effective amount of the Wee1 inhibitor (e.g., AZD-1775, though it is preferably at most 60 mg/kg twice daily or 120 mg/kg) is at least about 100 mg to 500 mg per kg of the subject's body weight (e.g., at least about 100, 125, 150, 175, 200, 225, 250, 275, 300, 325, 350, 375, 400, 425, 450, 475, 500, or more mg per kg of the subject's body weight). In some embodiments, the therapeutically effective amount of the Wee1 inhibitor (e.g., AZD-1775) is about 4 mg per kg of the subject's body weight. In some embodiments, the therapeutically effective amount of the Wee1 inhibitor (e.g., AZD-1775) is about 15 mg per kg of the subject's body weight. In some other embodiments, the therapeutically effective amount of the Wee1 inhibitor (e.g., AZD-1775) is about 30 mg per kg of the subject's body weight.

In some embodiments, a dose of the Wee1 inhibitor (e.g., AZD-1775) comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of the Wee1 inhibitor. In other embodiments, a dose of the Wee1 inhibitor (e.g., AZD-1775) comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of the Wee1 inhibitor.

In particular embodiments, a dose of the Wee1 inhibitor (e.g., AZD-1775) comprises between about 100 mg and 400 mg (e.g., about 100, 125, 150, 175, 200, 225, 250, 275, 300, 325, 350, 375, or 400 mg) of the Wee1 inhibitor. In some embodiments, a dose of the Wee1 inhibitor comprises about 225 mg of the Wee1 inhibitor.

In some embodiments, a dose of the Wee1 inhibitor (e.g., AZD-1775) is at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of the Wee1 inhibitor.

In some embodiments, a dose of the Wee1 inhibitor (e.g., AZD-1775) contains a therapeutically effective amount of the Wee1 inhibitor. In other embodiments, a dose of the Wee1 inhibitor (e.g., AZD-1775) contains less than a therapeutically effective amount of the Wee1 inhibitor if the Wee1 inhibitor were administered without a Chk1 inhibitor.

In some embodiments, the method further comprises administering to the subject a therapeutically effective amount of a third, DNA-damaging agent to increase the efficacy of the Chk1 and Wee1 inhibitors. In some embodiments, the DNA-damaging agent is an antimetabolite (e.g., capecitabine, 5-fluorouracil, gemcitabine, or pemetrexed), a topoisomerase poison or inhibitor (e.g., camptothecin or etoposide), an alkylating agent (e.g., a nitrogen mustard, a nitrosourea, temozolomide, or S23906), or a crosslinking drug (e.g., cisplatin, carboplatin, oxaliplatin, or mitomycin C).

In some embodiments, the therapeutically effective amount of the DNA-damaging agent is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In other embodiments, the therapeutically effective amount of the DNA-damaging agent is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, the therapeutically effective amount of the DNA-damaging agent is at least about 100 mg to 500 mg per kg of the subject's body weight (e.g., at least about 100, 125, 150, 175, 200, 225, 250, 275, 300, 325, 350, 375, 400, 425, 450, 475, 500, or more mg per kg of the subject's body weight).

In some embodiments, a dose of the DNA-damaging agent comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of the Wee1 inhibitor. In other embodiments, a dose of the DNA-damaging agent comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of the DNA-damaging agent.

In particular embodiments, a dose of the DNA-damaging agent comprises between about 100 mg and 400 mg (e.g., about 100, 125, 150, 175, 200, 225, 250, 275, 300, 325, 350, 375, or 400 mg) of the third, DNA-damaging agent.

In some embodiments, a dose of the DNA-damaging agent is at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of the Wee1 inhibitor.

In some embodiments, a dose of the DNA-damaging agent contains a therapeutically effective amount of the DNA-damaging agent. In other embodiments, a dose of the DNA-damaging agent contains less than a therapeutically effective amount of the DNA-damaging agent if the DNA-damaging agent were administered without a Chk1 and Wee1 inhibitor.

The data obtained from, for example, animal studies (e.g., rodents and monkeys) can be used to formulate a dosage range for use in humans. The dosage of compounds of the present invention lies preferably within a range of circulating concentrations that include the ED₅₀ with little or no toxicity. The dosage can vary within this range depending upon the dosage form employed and the route of administration. For any composition (e.g., comprising a Chk1 inhibitor, such as Compound 1; a Wee1 inhibitor, such as AZD-1775; or a combination of a Chk1 inhibitor and a Wee1 inhibitor) for use in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

It is furthermore understood that appropriate doses of a composition (e.g., comprising a Chk1 inhibitor, such as Compound 1; a Wee1 inhibitor, such as AZD-1775; or a combination of a Chk1 inhibitor and a Wee1 inhibitor) depend upon the potency of the composition with respect to the desired effect to be achieved. When one or more of these compositions is to be administered to a mammal, a physician, veterinarian, or researcher may, for example, prescribe a relatively low dose at first, subsequently increasing the dose until an appropriate response is obtained. In addition, it is understood that the specific dose level for any particular mammal subject will depend upon a variety of factors including the activity of the specific composition employed; the age, body weight, general health, sex, and diet of the subject; the time of administration; the route of administration; the rate and mode of excretion; effects of any drug combinations; and the degree of expression or activity to be modulated.

In certain embodiments, a combination of a Chk1 inhibitor (e.g., Compound 1) and a Wee1 inhibitor (e.g., AZD-1775) is administered to the subject. When the Chk1 inhibitor (e.g., Compound 1) and the Wee1 inhibitor (e.g., AZD-1775) are co-administered to the subject, the Chk1 inhibitor and the Wee1 inhibitor can either be administered simultaneously or sequentially.

In certain embodiments, the DNA-damaging agent is also administered to the subject. When the DNA-damaging agent is co-administered to the subject, the Chk1 inhibitor and the Wee1 inhibitor can either be administered simultaneously or sequentially with the DNA-damaging agent as well.

In some embodiments, both the Chk1 inhibitor (e.g., Compound 1) and the Wee1 inhibitor (e.g., AZD-1775) are administered at the same time. In other embodiments, the Chk1 inhibitor (e.g., Compound 1) and the Wee1 inhibitor (e.g., AZD-1775) are not administered at the same time but are administered the same number of times per day, or the same number of times per week, or the same number of times per month (e.g., both are adminsitered once per day, twice per day, once per week, twice per week, and so on). In some other embodiments, the Chk1 inhibitor (e.g., Compound 1) and the Wee1 inhibitor (e.g., AZD-1775) are given on different dosing schedules. As a non-limiting example, the Chk1 inhibitor is administered once per day, and the Wee1 inhibitor is adminsitered twice per day, or vice versa. As another non-limiting example, the Chk1 inhibitor is administered once per day, and the Wee1 inhiibtor is administered once every 2, 3, 4, 5, 6, or more days, or vice versa. The skilled artisan will also appreciate that certain factors may influence the dosage and timing required to effectively treat a subject, including but not limited to the severity of the disease or malignant condition, previous treatments, the general health or age of the subject, and other diseases present. Moreover, treatment of a subject with a therapeutically effective amount of a composition (e.g., comprising a Chk1 inhibitor, such as Compound 1; a Wee1 inhibitor, such as AZD-1775; or a combination of a Chk1 inhibitor and a Wee1 inhibitor) can include a single treatment or, preferably, can include a series of treatments.

Optimum dosages, toxicity, and therapeutic efficacy of the compositions (e.g., comprising a Chk1 inhibitor, such as Compound 1; a Wee1 inhibitor, such as AZD-1775; or a combination of a Chk1 inhibitor and a Wee1 inhibitor that is administered according to the methods of the present invention may vary depending on the relative potency of the administered composition and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

Optimal dosing schedules can be calculated from measurements of active ingredient accumulation in the body of a subject. In general, dosage is from about 1 ng to about 1,000 mg per kg of body weight and may be given once or more daily, weekly, monthly, or yearly. Persons of ordinary skill in the art can easily determine optimum dosages, dosing methodologies and repetition rates. One of skill in the art will be able to determine optimal dosing for administration of a Chk1 inhibitor or a combination of a Chk1 inhibitor and a Wee1 inhibitor to a human being following established protocols known in the art and the disclosure herein.

Whether the Chk1 inhibitor (e.g., Compound 1) and the Wee1 inhibitor (e.g., AZD-1775) are administered simultaneously or sequentially, the doses of the Chk1 inhibitor and the Wee1 inhibitor can be any dose described herein. In some embodiments, the doses of the Chk1 inhibitor and the Wee1 inhibitor are therapeutically effective amounts. In other embodiments, the dose of the Chk1 inhibitor is a therapeutically effective amount and the dose of the Wee1 inhibitor is less than a therapeutically effective amount (i.e., one or more subsequent doses of the Wee1 inhibitor are administered in order for the therapeutically effective amount to be delivered to the subject). In some other embodiments, the dose of the Wee1 inhibitor is a therapeutically effective amount and the dose of the Chk1 inhibitor is less than a therapeutically effective amount (i.e., one or more subsequent doses of the Chk1 inhibitor are administered in order for the therapeutically effective amount to be delivered to the subject). In some embodiments, the dose of Compound 1 is about 7.5 mg, 12.5 mg, 20 mg, 25 mg, or 50 mg per kg of the subject's body weight, and the dose of AZD-1775 is about 15 mg or 30 mg per kg of the subject's body weight.

When the Chk1 inhibitor (e.g., Compound 1) and the Wee1 inhibitor (e.g., AZD-1775) are simultaneously co-administered to the subject, the Chk1 inhibitor and the Wee1 inhibitor can be administered by the same route, or by different routes. As a non-limiting example, the Chk1 inhibitor and the Wee1 inhibitor can simultaneously be administered orally. As another non-limiting example, the Chk1 inhibitor can be administered orally, and the Wee1 inhibitor can be administered at the same time by another route (e.g., intravenously, intramuscularly, subcutaneously, intratumorally, or intraperitoneally). Alternatively, the Wee1 inhibitor can be administered orally, and the Chk1 inhibitor can be administered at the same time by another route (e.g., intravenously, intramuscularly, subcutaneously, intratumorally, or intraperitoneally).

For sequential co-administration, the Chk1 inhibitor (e.g., Compound 1) can be administered before the Wee1 inhibitor (e.g., AZD-1775), or vice versa. In some embodiments, the Chk1 inhibitor and the Wee1 inhibitor are administered by the same route, but administration of the Chk1 inhibitor and the Wee1 inhibitor are separated by some amount of time. In other embodiments, the Chk1 inhibitor and the Wee1 inhibitor are administered by different routes, and administration of the Chk1 inhibitor and the Wee1 inhibitor are separated by some amount of time. As a non-limiting example, the Chk1 inhibitor is administered orally, and the Wee1 inhibitor is subsequently administered orally sometime later, or vice versa. As another non-limiting example, the Chk1 inhibitor is administered orally, and the Wee1 inhibitor is subsequently administered by another route (e.g., intravenously, intramuscularly, subcutaneously, intratumorally, or intraperitoneally) sometime later, or vice versa.

For sequential co-administration, one of skill in the art will readily be able to determine the appropriate amount of time between administration of the Chk1 inhibitor (e.g., Compound 1) and the Wee1 inhibitor (e.g., AZD-1775). In some embodiments, administration of the Chk1 inhibitor and the Wee1 inhibitor are separated by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 50, 51, 52, 53, 54, 55, 56, 57, 58, 59, 60, or more minutes. In other embodiments, administration of the Chk1 inhibitor and the Wee1 inhibitor are separated by about 1, 2, 3, 4, 5, 6, 7, 8, 9, 10, 11, 12, 13, 14, 15, 16, 17, 18, 19, 20, 21, 22, 23, 24, or more hours. In some other embodiments, administration of the Chk1 inhibitor and the Wee1 inhibitor are separated by about 1, 2, 3, 4, 5, 6, 7, or more days. In yet other embodiments, administration of the Chk1 inhibitor and the Wee1 inhibitor are separated by about 1, 2, 3, 4, 5, 6, 7, 8, 9, 10, 11, 12, or more weeks. In some embodiments, administration of the Chk1 inhibitor and the Wee1 inhibitor are separated by about 1, 2, 3, 4, 5, 6, 7, 8, 9, 10, 11, 12, 13, 14, 15, 16, 17, 18, 19, 20, 21, 22, 23, 24, or more months.

In some embodiments, the Chk1 inhibitor, the Wee1 inhibitor, or the combination of a Chk1 inhibitor and a Wee1 inhibitor is administered 1, 2, 3, 4, 5, or more times per day. In other embodiments, Chk1 inhibitor, the Wee1 inhibitor, or the combination of a Chk1 inhibitor and a Wee1 inhibitor is administered 1, 2, 3, 4, 5, 6, 7, or more times per week. In some other embodiments, Chk1 inhibitor, the Wee1 inhibitor, or the combination of a Chk1 inhibitor and a Wee1 inhibitor is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more times per month.

In some embodiments, Chk1 inhibitor, the Wee1 inhibitor, or the combination of a Chk1 inhibitor and a Wee1 inhibitor is administered about every 1, 2, 3, 4, 5, 6, 7, 8, 9, 10, or more days. In other embodiments, the Chk1 inhibitor, the Wee1 inhibitor, or the combination of a Chk1 inhibitor and a Wee1 inhibitor is administered about every 1, 2, 3, 4, or more weeks. In some other embodiments, the Chk1 inhibitor, the Wee1 inhibitor, or the combination of a Chk1 inhibitor and a Wee1 inhibitor is administered about every 1, 2, 3, 4, 5, 6, 7, 8, 8, 10, 11, 12, or more months.

Following successful treatment, it may be desirable to have the subject undergo maintenance therapy to prevent the recurrence of the cancer (e.g., acute myeloid leukemia, esophageal cancer, gastric cancer, mantle cell lymphoma, non-small cell lung cancer (NSCLC), ovarian cancer, head and neck cancer, liver cancer, pancreatic cancer, prostate cancer, or a central nervous system cancer).

Determination of an effective amount is well within the capability of those skilled in the art, especially in light of the detailed disclosure provided herein. Generally, an efficacious or effective amount of a composition (e.g., comprising a Chk1 inhibitor, a Wee1 inhibitor, or a combination of a Chk1 inhibitor and a Wee1 inhibitor) is determined by first administering a low dose or small amount of the composition, and then incrementally increasing the administered dose or dosages, until a desired effect of is observed in the treated subject with minimal or no toxic side effects.

Single or multiple administrations of a composition (e.g., comprising a Chk1 inhibitor, such as Compound 1; a Wee1 inhibitor, such as AZD-1775; or a combination of a Chk1 inhibitor and a Wee1 inhibitor) are administered depending on the dosage and frequency as required and tolerated by the patient. In any event, the composition should provide a sufficient quantity of the composition to effectively treat the patient. Generally, the dose is sufficient to prevent, treat, or ameliorate symptoms or signs of disease without producing unacceptable toxicity to the patient.

In some embodiments, treating the subject comprises inhibiting cancer (e.g., acute myeloid leukemia, esophageal cancer, gastric cancer, mantle cell lymphoma, non-small cell lung cancer (NSCLC), ovarian cancer, head and neck cancer, liver cancer, pancreatic cancer, prostate cancer, or a central nervous system cancer) cell growth, inhibiting cancer cell proliferation, inhibiting cancer cell migration, inhibiting cancer cell invasion, decreasing or eliminating one or more signs or symptoms of cancer, reducing the size (e.g., volume) of a cancer tumor, reducing the number of cancer tumors, reducing the number of cancer cells, inducing cancer cell necrosis, pyroptosis, oncosis, apoptosis, autophagy, or other cell death, increasing survival time of the subject, or enhancing the therapeutic effects of another drug or therapy. In particular embodiments, the subject does not have cancer.

### B. Pharmaceutical Compositions

In another aspect, the present disclosure provides a pharmaceutical composition comprising a Chk1 inhibitor and a pharmaceutically acceptable carrier. In some embodiments, the Chk1 inhibitor is one disclosed in PCT Application Publication No. WO 2015/120390. In some embodiments, the Chk1 inhibitor is Compound 1.

In some embodiments, the pharmaceutical composition further comprises a Wee1 inhibitor. In particular embodiments, the Wee1 inhibitor is selected from the group consisting of a pyrazolopyrimidine derivative, a pyridopyrimidine, 4-(2-chlorophenyl)-9-hydroxypyrrolo[3,4-c]carbazole-1,3-(2H,6H)-dione, 6-butyl-4-(2-chlorophenyl)-9-hydroxypyrrolo[3,4-c]carbazole-1,3-(2H,6H)-dione, 4-(2-phenyl)-9-hydroxypyrrolo[3,4-c]carbazole-1,3-(2H,6H)-dione, an anti-Wee1 antibody, and an anti-Wee1 small interfering RNA (siRNA) molecule. In some embodiments, the pyridopyrimidine is pyrido [2,3-d] pyrimidine. In particular embodiments, the pyrazolopyrimidine derivative is AZD-1775.

In some embodiments, the Chk1 inhibitor (e.g., Compound 1) is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, the Chk1 inhibitor is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, the Chk1 inhibitor is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, the Chk1 inhibitor is present at a concentration at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

In some embodiments, the Wee1 inhibitor (e.g., AZD-1775) is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, the Wee1 inhibitor is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, the Wee1 inhibitor is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, the Wee1 inhibitor is present at a concentration of at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

In some embodiments, the DNA-damaging agent is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, the DNA-damaging agent is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, the DNA-damaging agent is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, the DNA-damaging agent is present at a concentration of at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

The pharmaceutical compositions of the present invention may be prepared by any of the methods well-known in the art of pharmacy. Pharmaceutically acceptable carriers suitable for use with the present invention include any of the standard pharmaceutical carriers, buffers and excipients, including phosphate-buffered saline solution, water, and emulsions (such as an oil/water or water/oil emulsion), and various types of wetting agents or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent.

The pharmaceutical compositions of the present invention can include a Chk1 inhibitor, such as Compound 1; a Wee1 inhibitor, such as AZD-1775; or a combination of a Chk1 inhibitor and a Wee1 inhibitor, or any pharmaceutically acceptable salts thereof, as active ingredients and a pharmaceutically acceptable carrier or excipient or diluent. A pharmaceutical composition may optionally contain other therapeutic ingredients.

The compositions (e.g., a Chk1 inhibitor, such as Compound 1; a Wee1 inhibitor, such as AZD-1775; or a combination of a Chk1 inhibitor and a Wee1 inhibitor) can be combined as the active ingredients in intimate admixture with a suitable phrmaceutical carrier or excipient according to conventional pharmaceutical compounding techniques. Any carrier or excipient suitable for the form of preparation desired for administration is contemplated for use with the compounds disclosed herein.

The pharmaceutical compositions include those suitable for oral, topical, parenteral, pulmonary, nasal, or rectal administration. The most suitable route of administration in any given case will depend in part on the nature and severity of the cancer condition and also optionally the stage of the cancer.

Other pharmaceutical compositions include those suitable for systemic (e.g., enteral or parenteral) administration. Systemic administration includes oral, rectal, sublingual, or sublabial administration. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In particular embodiments, pharmaceutical compositions of the present invention may be administered intratumorally.

Compositions for pulmonary administration include, but are not limited to, dry powder compositions consisting of the powder of a compound described herein (e.g., a Chk1 inhibitor, such as Compound 1; a Wee1 inhibitor, such as AZD-1775; or a combination of a Chk1 inhibitor and a Wee1 inhibitor), or a salt thereof, and the powder of a suitable carrier or lubricant. The compositions for pulmonary administration can be inhaled from any suitable dry powder inhaler device known to a person skilled in the art.

Compositions for systemic administration include, but are not limited to, dry powder compositions consisting of the composition as set forth herein (e.g., a Chk1 inhibitor, such as Compound 1; a Wee1 inhibitor, such as AZD-1775; or a combination of a Chk1 inhibitor and a Wee1 inhibitor) and the powder of a suitable carrier or excipient. The compositions for systemic administration can be represented by, but not limited to, tablets, capsules, pills, syrups, solutions, and suspensions.

In some embodiments, the compositions (e.g., a Chk1 inhibitor, such as Compound 1; a Wee1 inhibitor, such as AZD-1775; or a combination of a Chk1 inhibitor and a Wee1 inhibitor) further include a pharmaceutical surfactant. In other embodiments, the compositions further include a cryoprotectant. In some embodiments, the cryoprotectant is selected from the group consisting of glucose, sucrose, trehalose, lactose, sodium glutamate, PVP, HPβCD, CD, glycerol, maltose, mannitol, and saccharose.

Pharmaceutical compositions or medicaments for use in the present invention can be formulated by standard techniques using one or more physiologically acceptable carriers or excipients. Suitable pharmaceutical carriers are described herein and in Remington: The Science and Practice of Pharmacy, 21st Ed., University of the Sciences in Philadelphia, Lippencott Williams & Wilkins (2005).

Controlled-release parenteral formulations of the compositions (e.g., a Chk1 inhibitor, such as Compound 1; a Wee1 inhibitor, such as AZD-1775; or a combination of a Chk1 inhibitor and a Wee1 inhibitor) can be made as implants, oily injections, or as particulate systems. For a broad overview of delivery systems see Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995), which is incorporated herein by reference. Particulate systems include microspheres, microparticles, microcapsules, nanocapsules, nanospheres, and nanoparticles.

Polymers can be used for ion-controlled release of compositions of the present invention. Various degradable and nondegradable polymeric matrices for use in controlled drug delivery are known in the art (Langer R., Accounts Chem. Res., 26:537-542 (1993)). For example, the block copolymer, polaxamer 407 exists as a viscous yet mobile liquid at low temperatures but forms a semisolid gel at body temperature. It has shown to be an effective vehicle for formulation and sustained delivery of recombinant interleukin 2 and urease (Johnston et al., Pharm. Res., 9:425-434 (1992); and Pec et al., J. Parent. Sci. Tech., 44(2):58 65 (1990)). Alternatively, hydroxyapatite has been used as a microcarrier for controlled release of proteins (Ijntema et al., Int. J. Pharm., 112:215-224 (1994)). In yet another aspect, liposomes are used for controlled release as well as drug targeting of the lipid-capsulated drug (Betageri et al., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

For oral administration of a Chk1 inhibitor (e.g., Compound 1), a Wee1 inhibitor (e.g., AZD-1775), or a combination of a Chk1 inhibitor and a Wee1 inhibitor, a pharmaceutical composition or a medicament can take the form of, for example, a tablet or a capsule prepared by conventional means with a pharmaceutically acceptable excipient. The present invention provides tablets and gelatin capsules comprising a Chk1 inhibitor, a Wee1 inhibitor, or a combination of a Chk1 inhibitor and a Wee1 inhibitor, or a dried solid powder of these drugs, together with (a) diluents or fillers, e.g., lactose, dextrose, sucrose, mannitol, sorbitol, cellulose (e.g., ethyl cellulose, microcrystalline cellulose), glycine, pectin, polyacrylates or calcium hydrogen phosphate, calcium sulfate, (b) lubricants, e.g., silica, talcum, stearic acid, magnesium or calcium salt, metallic stearates, colloidal silicon dioxide, hydrogenated vegetable oil, corn starch, sodium benzoate, sodium acetate or polyethyleneglycol; for tablets also (c) binders, e.g., magnesium aluminum silicate, starch paste, gelatin, tragacanth, methylcellulose, sodium carboxymethylcellulose, polyvinylpyrrolidone or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, or sweetening agents as appropriate. If desired, preparations for oral administration can be suitably formulated to give controlled release of the active compound(s).

Typical formulations for topical administration of Chk1 inhibitor, the Wee1 inhibitor, or the combination of a Chk1 inhibitor and a Wee1 inhibitor include creams, ointments, sprays, lotions, and patches. The pharmaceutical composition can, however, be formulated for any type of administration, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral or rectal administration is also contemplated.

Suitable formulations for transdermal application include an effective amount of one or more compounds described herein, optionally with a carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

The compositions and formulations set forth herein (e.g., comprising a Chk1 inhibitor, a Wee1 inhibitor, or the combination of a Chk1 inhibitor and a Wee1 inhibitor) can be formulated for parenteral administration by injection, for example by bolus injection or continuous infusion. Formulations for injection can be presented in unit dosage form, for example, in ampules or in multi-dose containers, with an added preservative. Injectable compositions are preferably aqueous isotonic solutions or suspensions, and suppositories are preferably prepared from fatty emulsions or suspensions. The compositions may be sterilized or contain adjuvants, such as preserving, stabilizing, wetting or emulsifying agents, solution promoters, salts for regulating the osmotic pressure or buffers. Alternatively, the active ingredient(s) can be in powder form for constitution with a suitable vehicle, for example, sterile pyrogen-free water, before use. In addition, they may also contain other therapeutically valuable substances. The compositions are prepared according to conventional mixing, granulating or coating methods, respectively.

For administration by inhalation, the compositions (e.g., comprising a Chk1 inhibitor, a Wee1 inhibitor, or the combination of a Chk1 inhibitor and a Wee1 inhibitor) may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compositions (e.g., comprising a Chk1 inhibitor, a Wee1 inhibitor, or the combination of a Chk1 inhibitor and a Wee1 inhibitor) can also be formulated in rectal compositions, for example, suppositories or retention enemas, for example, containing conventional suppository bases, for example, cocoa butter or other glycerides.

Furthermore, the active ingredient(s) can be formulated as a depot preparation. Such long-acting formulations can be administered by implantation (for example, subcutaneously or intramuscularly) or by intramuscular injection. Thus, for example, one or more of the compounds described herein can be formulated with suitable polymeric or hydrophobic materials (for example as an emulsion in an acceptable oil) or ion exchange resins, or as sparingly soluble derivatives, for example, as a sparingly soluble salt.

### C. Kits

In another aspect, the present invention provides a kit for preventing or treating cancer in a subject, the kit comprising a pharmaceutical composition of the present invention (e.g., a pharmaceutical composition comprising a Chk1 inhibitor (e.g., Compound 1 or another Chk1 inhibitor described herein), a Wee1 inhibitor as described herein, or a pharmaceutical composition comprising a Chk1 inhibitor and a Wee1 inhibitor (e.g., AZD-1775 or another Wee1 inhibitor described herein).

The kits are suitable for preventing or treating any number of cancers. In some embodiments, the type of cancer that is prevented or treatment is selected from the group consisting of gastric cancer, head and neck cancer, a lung cancer (e.g., non-small cell lung cancer (NSCLC)), ovarian cancer, breast cancer, colorectal cancer, a nervous system cancer (e.g., central nervous system cancers), adrenal gland cancer, bladder cancer, a blood cancer (e.g., leukemia, acute myeloid leukemia, mantle cell lymphoma, anaplastic large cell lymphoma, B-cell acute lymphoblastic leukemia, Burkitt lymphoma, chronic lymphocytic leukemia, chronic myelogenous leukemia, multiple myeloma, acute promyelocytic leukemia, T-cell acute lymphoblastic leukemia), bone cancer, cervical cancer, esophageal cancer, eye cancer, renal cancer, liver cancer, muscle cancer, nasal cancer, pancreatic cancer, pharyngeal cancer, placental cancer, prostate cancer, skin cancer, soft tissue cancers, submaxillary gland cancer, thyroid cancer, tongue cancer, and uterine cancer. In particular embodiments, the cancer that is prevented or treated is selected from the group consisting of acute myeloid leukemia, esophageal cancer, gastric cancer, mantle cell lymphoma, non-small cell lung cancer (NSCLC), ovarian cancer, head and neck cancer, liver cancer, pancreatic cancer, prostate cancer, and a central nervous system cancer. In some embodiments, the cancer is a metastatic cancer. In other embodiments, the cancer is an advanced cancer. In some other embodiments, the cancer is a drug-resistant cancer. In some embodiments, the cancer is a multidrug-resistant cancer. In some embodiments, the cancer is advanced, metastatic, or drug-resistant. In some embodiments, the cancer is mantle cell lymphoma. In some embodiments, the cancer is mantle cell lymphoma and the subject has a chromosomal translocation at t(11;14)(q13;q32). In particular embodiments, the cancer is a breast cancer or metastatic breast cancer.

Materials and reagents to carry out the various methods of the present invention can be provided in kits to facilitate execution of the methods. As used herein, the term “kit” includes a combination of articles that facilitates a process, assay, analysis, or manipulation. In particular, kits of the present invention find utility in a wide range of applications including, for example, diagnostics, prognostics, therapy, and the like.

Kits can contain chemical reagents as well as other components. In addition, the kits of the present invention can include, without limitation, instructions to the kit user, apparatus and reagents for administering Chk1 inhibitors (e.g., Compound 1), Wee1 inhibitors (e.g., AZD-1775), or pharmaceutical compositions thereof or combinations of Chk1 inhibitors and Wee1 inhibitors or pharmaceutical compositions thereof, sample tubes, holders, trays, racks, dishes, plates, solutions, buffers, or other chemical reagents. Kits of the present invention can also be packaged for convenient storage and safe shipping, for example, in a box having a lid.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

The examples provided herein highlight the activity of the orally bioavailable, selective small molecule Chk1 inhibitor Compound 1 in solid and hematological tumor-derived cell lines. Compound 1 is a sub-nanomolar enzymatic inhibitor of Chk1 with limited off-target activity against a panel of protein kinases. When evaluated in large cell line panels in vitro, Compound 1 demonstrated a broad potency range as a single agent in solid and hematological tumor-derived cells lines, with IC₅₀ values ranging from 30 nM to greater than 50 μM.

Several solid tumor types demonstrated enriched sensitivity to Compound 1 in vitro, including gastric, non-small cell lung, and ovarian cancers. Treatment of sensitive cell lines with Compound 1 resulted in the induction of DNA damage, as measured by phosphorylated histone H2AX, and the induction of cell death. Compound 1 was active as a single agent in SK-MES-1 and NCI-H727 NSCLC tumor xenograft models in vivo with minimal effects on body weight in treated mice. In addition to the potent single-agent activity of Compound 1, combination with the Wee1 inhibitor AZD-1775 was highly synergistic in vitro in multiple solid tumor cell lines and the combination was more efficacious than either agent alone in NSCLC tumor xenograft models.

In addition, several hematological tumor types demonstrated enriched sensitivity to Compound 1 in vitro and in vivo. Compound 1 demonstrated compelling single-agent activity on mantle cell lymphoma (MCL) and acute myeloid leukemia (AML) cell lines in vitro and in vivo, including complete tumor regression in a Jeko-1 xenograft model. Furthermore, Compound 1 showed strong anti-proliferative activity and induction of DNA damage in AML-derived cell lines, as well as single-agent activity in an MV-411 tumor xenograft model.

The experimental results presented in these examples demonstrate that Compound 1 is a highly potent and selective Chk1 inhibitor. In particular, Compound 1 exhibited sub-nanomolar potency against Chk1, with limited off-target kinase activity (i.e., more than 1,000× selective for Chk1 than for Chk2). Furthermore, Compound 1 demonstrated attractive pharmaceutical properties such as oral bioavailability and a low efflux ratio (allowing for a flexible dose schedule and the treatment of multiple drug-resistant and CNS metastasized cancers), good metabolic stability, no CYP inhibition liabilities, an excellent hERG inhibition index, and a low risk of cardiotoxicity (based on cynomolgus monkey safety pharmacology study results). In addition, not only did Compound 1 demonstrate potent activity as a single agent in multiple tumor models, but synergistic activity was observed in combination with a Wee1 inhibitor. Synergistic effects were observed in the induction of DNA damage, apoptosis, and tumor control. These data show that Compound 1 has clinical utility for the treatment of solid and hematological tumor diseases.

### Example 1

### Drug Properties of Compound 1, a Novel, Orally Available Checkpoint Kinase 1 Inhibitor

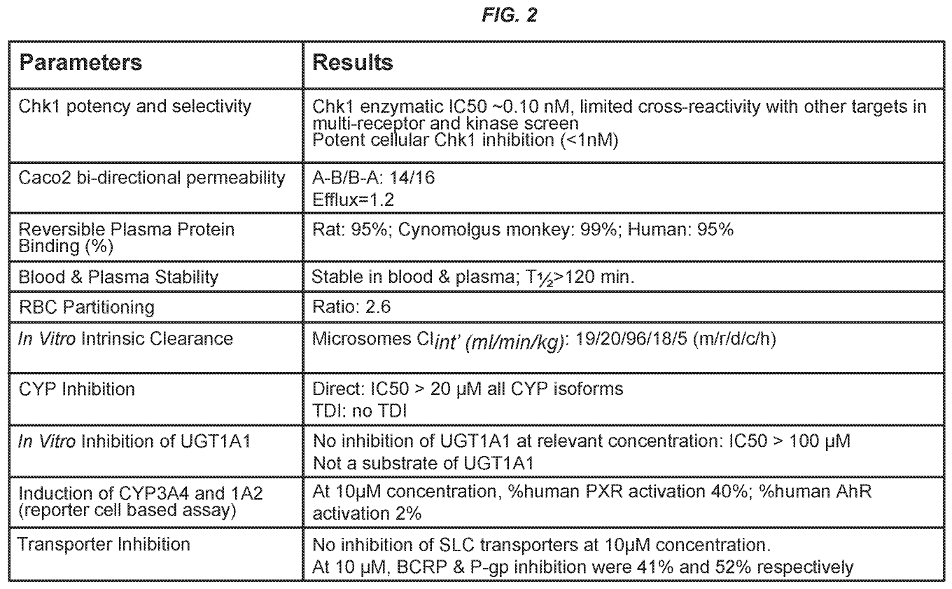

Compound 1 is a Chk1 inhibitor that exhibits many excellent drug properties, some of which are presented in FIG. 2. In particular, Compound 1 exhibits sub-nanomolar potency against Chk1, having limited off-target activities. In addition, Compound 1 displays favorable absorption, distribution, metabolism, and excretion (ADME) properties, pharmacokinetics, and oral bioavailability. 7-day repeat dose tolerability studies have been completed in mice, rats, and cynomolgus monkeys, and there have been no findings in a cynomolgus monkey GLP cardiovascular safety study (including corrected QT (QTc) interval, left ventricular pressure (LVP), and contractility end points). Compound 1 is active as a single agent, but is also active in combination with chemotherapeutic agents and Wee1 inhibitors.

### Example 2

### Selectivity and Potency of Compound 1

### Enzymatic Selectivity of Compound 1

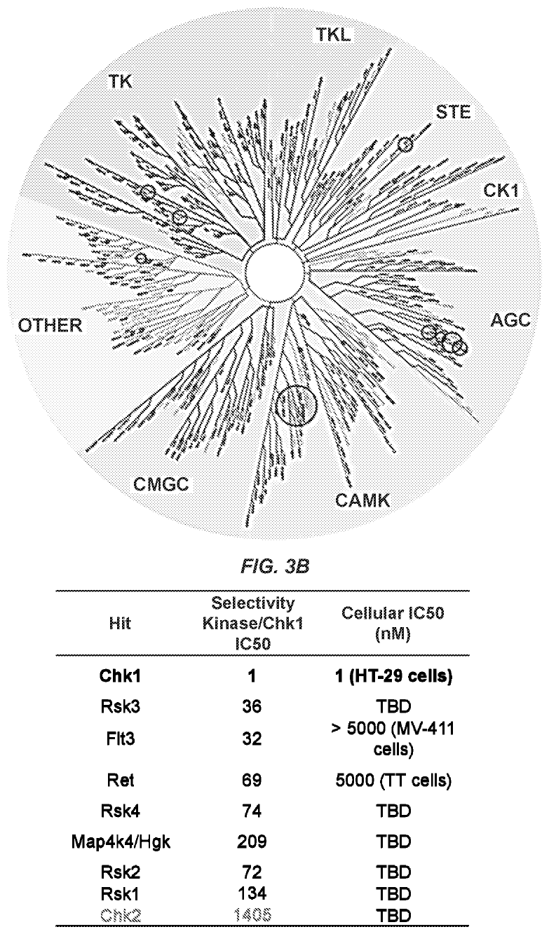

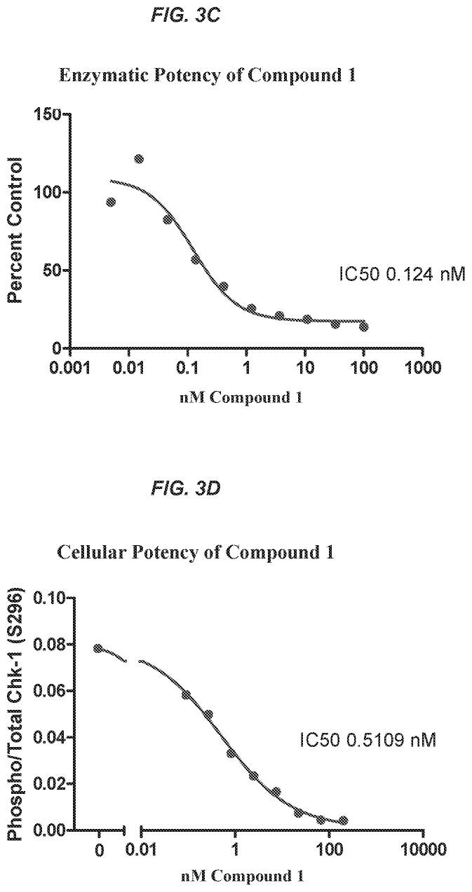

Compound 1 was screened against a panel of 120 kinases, including those represented in FIG. 3A, using a 1 μM ATP concentration. All kinases inhibited more than 80% and Chk2 are shown in FIG. 3B. The IC₅₀ values, measured at the ATP Kₘ for each kinase, are represented relative to Chk1 in FIG. 3B. Cellular IC₅₀ values were derived from signal transduction assays in relevant cell lines using phosphor-epitope-specific antibodies.

### Enzymatic and Cellular Potency of Compound 1

Enzymatic assays were performed using 10 μM of [γ-33P]-ATP and 20 μM of the peptide substrate KKKVSRSGLYRSPSMPENLNRPR (SEQ ID NO:1) that was obtained from Reaction Biology Corp. As can be seen in FIG. 3C, the IC₅₀ was 0.124 nM.

Cellular Chk1 was assayed using HT-29 colon carcinoma cells in an 18-hour assay by immunoblotting with a rabbit anti-Chk1 serine 296 phosphor-epitope antibody (obtained from Cell Signaling Technology Inc.). The results of this assay are shown in FIG. 3D. The IC₅₀ was 0.5109 nM.

### Example 3

### In Vitro Screening

Extensive screening against diverse cancer cell lines was performed to identify tumor types exhibiting sensitivity to Compound 1 as a single agent. A panel of 232 carcinoma derived cell lines was screened in a high-throughput proliferation assay using dilutions of Compound 1 or cisplatin. Cell lines were treated with serial half-log dilutions of Compound 1 or cisplatin using a starting concentration of 30 μM to achieve 9 dose levels and assayed 72 hours later for proliferation using a CellTiter-Glo® Assay (Promega). IC₅₀ (EC₅₀) values were calculated by fitting the dose-response data using a nonlinear regression model.

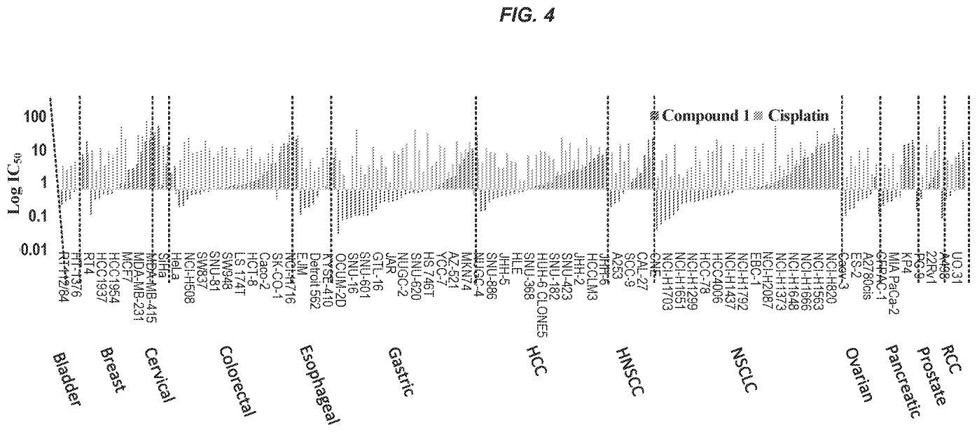

FIG. 4 shows that Compound 1 was effective in inhibiting growth in carcinoma cell lines derived from diverse histological origins. Furthermore, unique activity patterns were observed when comparing Compound 1 to cisplatin. Tumor types that were particularly sensitive to Compound 1 included esophageal cancer, gastric cancer, non-small cell lung cancer (NSCLC), ovarian cancer, and leukemia.

### Example 4

### In Vivo Screening in NSCLC Xenograft Models

In order to assess the in vivo efficacy of Compound 1, two different NSCLC xenograft models were used. SK-MES-1 or NCI-H727 tumor cells were inoculated subcutaneously in the flanks of athymic nude mice. Once tumors reached a volume of about 200 mm³, mice were randomized into study groups (n=10 per group). Mice were treated by oral gavage with Compound 1 at a dose of 12.5, 25, or 50 mg/kg once per day. A negative control group was also included, wherein the animals were administered vehicle only.

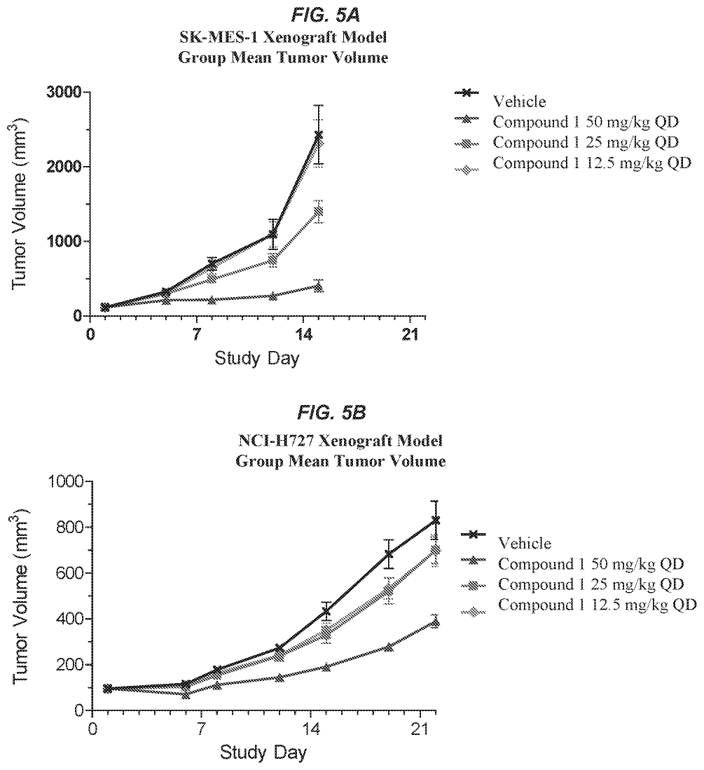

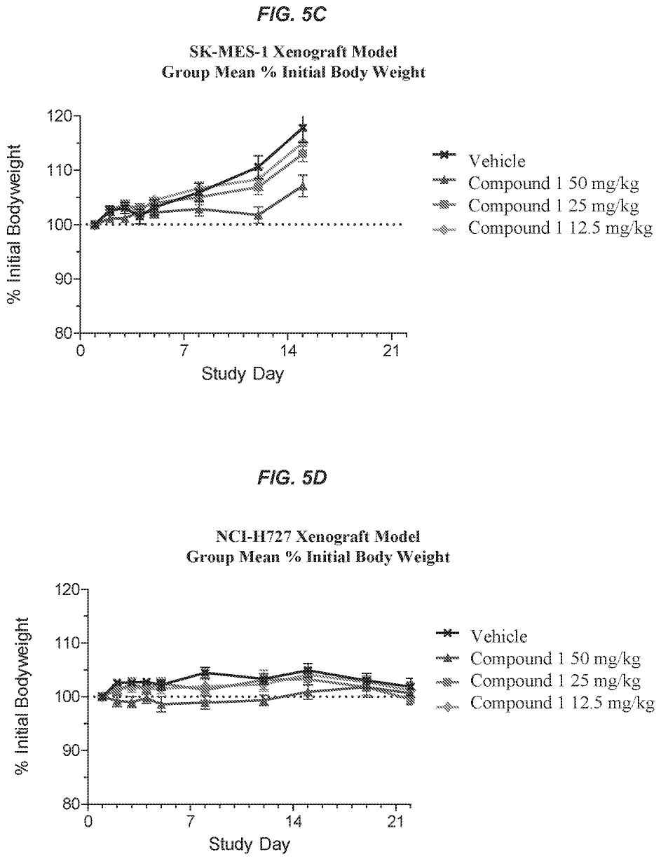

The effects on SK-MES-1 tumors are shown in FIGS. 5A and 5C, and the effects on NCI-H727 tumors are shown FIGS. 5B and 5D). As can be seen in FIGS. 5A-5D, Compound 1 inhibited tumor growth in both xenograft models in a dose-dependent manner.

### Example 5

### Cell Screening and Characterization of Compound 1 in Combination with a Wee1 Inhibitor

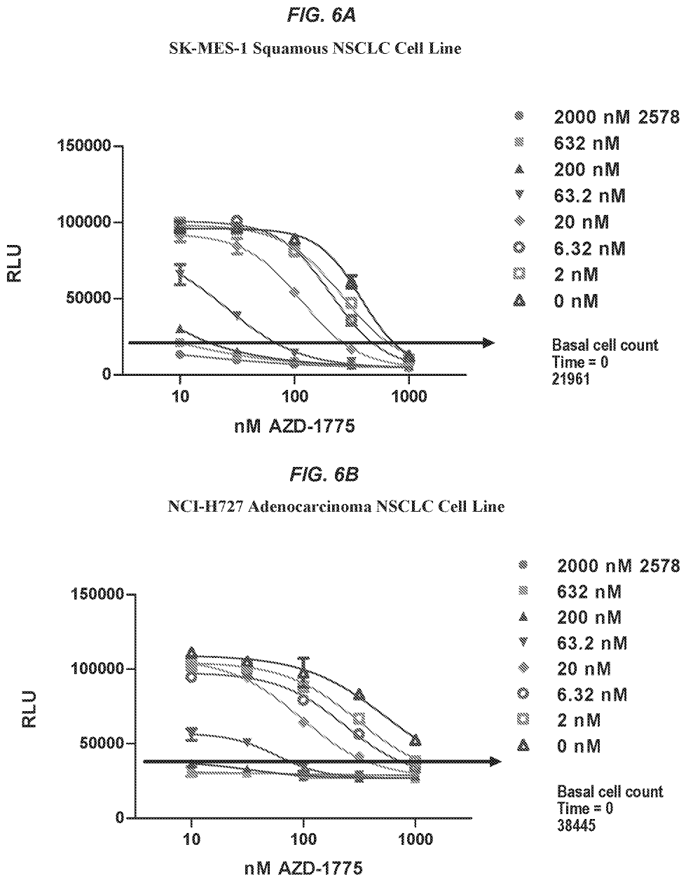

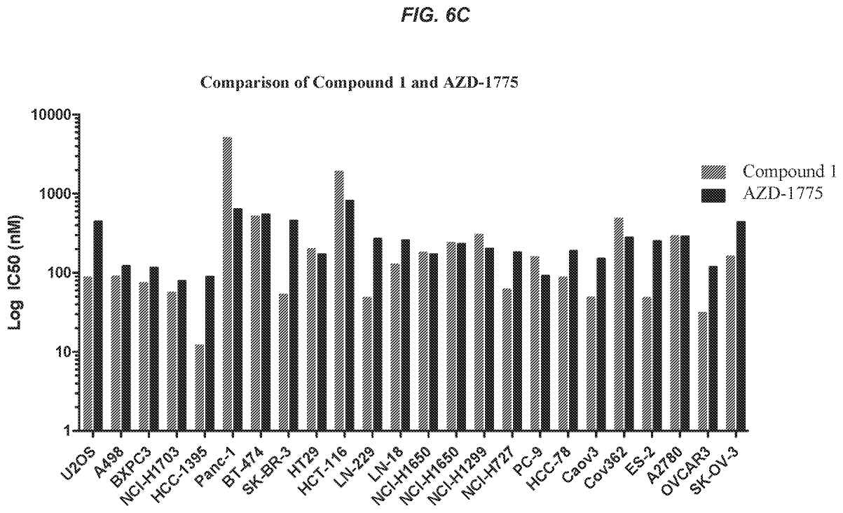

In addition to examining the efficacy of Compound 1 as a single agent, Compound 1 was tested in combination with the Wee1 inhibitor AZD-1775. SK-MES-1 and NCI-H727 tumor cells were treated with titrations of Compound 1 or the Wee-1 inhibitor AZD-1775, both alone and in combination. Cell proliferation was measured 72 hours after drug addition using a CellTiter-Glo® Assay (Promega). As can be seen in FIGS. 6A-6C, the combination of Compound 1 and AZD-1775 exhibited synergistic effects. Furthermore, unique patterns of activity were observed when the drugs were tested as single agents.

FIG. 6A depicts the results of cell viability assays that were performed using various combinations of Compound 1 and AZD-1775 to inhibit SK-MES NSCLC cancer cells. Cell viability was inhibited in a dose-dependent manner. Similarly, when various combinations of Compound 1 and AZD-1775 were tested against NCI-H727 NSCLC cells, dose-dependent inhibition of viability was observed (FIG. 6B). The IC₅₀ values of both drugs were tested in multiple cell lines, a comparison of which is presented in FIG. 6C.

### Example 6

### In Vivo Activity of Compound 1 in Combination with a Wee1 Inhibitor

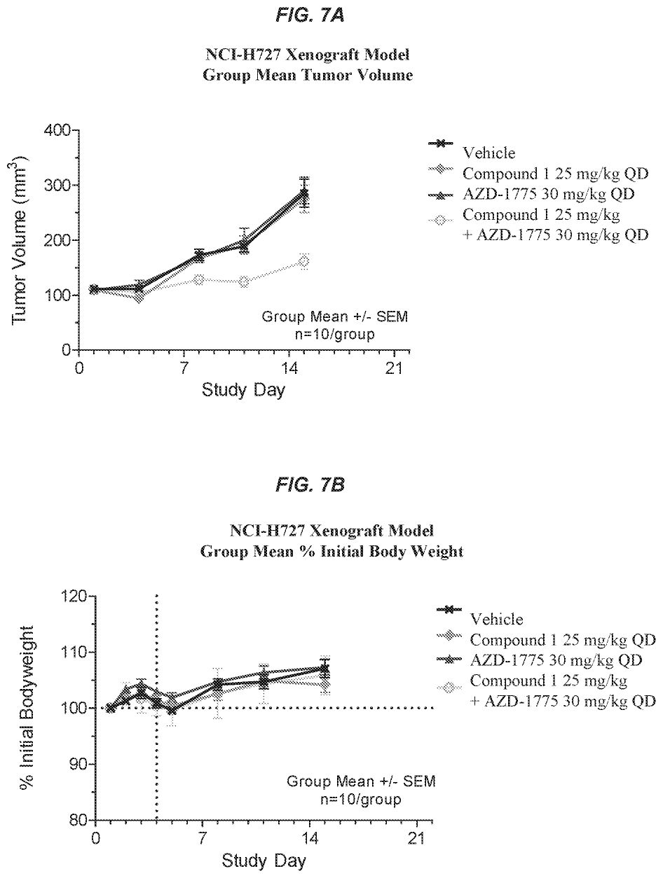

Using an NCI-H727 NSCLC tumor xenograft model, Compound 1 (25 mg/kg once per day) and AZD-11775 (30 mg/kg once per day), both individually and in combination, were tested for their ability to inhibit tumor size (FIGS. 7A and 7B). NCI-H727 tumor cells were inoculated subcutaneously in the flanks of athymic nude mice. One tumors reached a volume of about 200 mm³, mice were randomized into study groups (n=10 per group). Compound 1 and AZD-1775 were administered by oral gavage. A remarkable synergistic effect was observed when the two drugs were combined.

### Example 7

### High-Throughput in Vitro Screening of Hematological Tumor-Derived Cell Lines

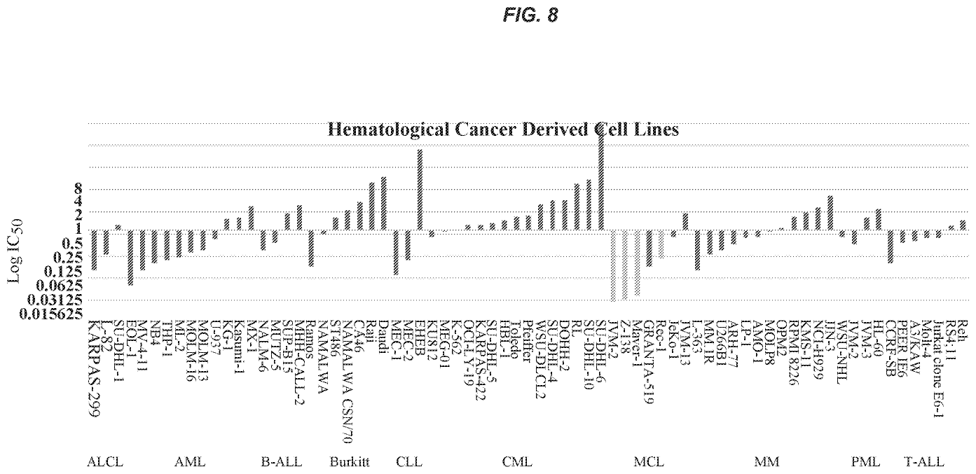

A panel of about 70 hematopoietic cell lines was screened for sensitivity to Compound 1 in a 72-hour proliferation assay (CrownBio Omnipanel). Cell lines that were screened included those representing anaplastic large cell lymphoma (ALCL), acute myeloid leukemia (AML), B-cell acute lymphoblastic leukemia (B-ALL), Burkitt lymphoma, chronic lymphocytic leukemia (CLL), chronic myelogenous leukemia (CML), mantle cell lymphoma (MCL), multiple myeloma (MM), acute promyelocytic leukemia (PML), and T-cell acute lymphoblastic leukemia (T-ALL). As shown in FIG. 8, subsets of the hematological tumor-derived cell lines that were particularly sensitive to inhibition by Compound 1 included MCL and AML cell lines.

### Example 8

### Anti-Proliferative Activity in MCL Cell Lines

Mantle cell lymphoma (MCL) is a rare and usually aggressive form of non-Hodgkin lymphoma that affects around 15,000 patients in the United States. The majority of MCL patients possess a chromosomal translocation at t(11;14)(q13;q32) that leads to the overexpression of cyclin D1. Since Chk1 and Wee1 kinases are regulators of Cdk/cyclin activity, MCL can be uniquely sensitized to Chk1 inhibitors alone or in combination with Wee1 inhibitors.

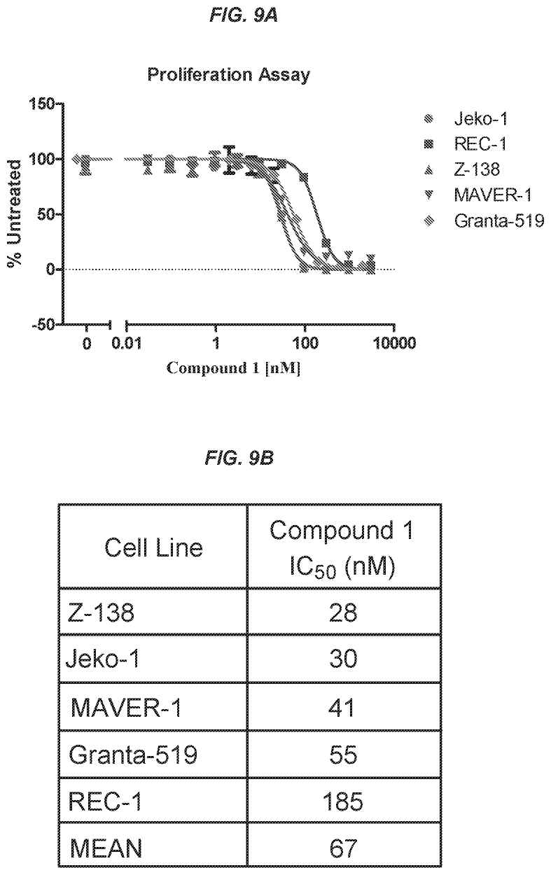

In order to assess the ability of Compound 1 to inhibit MCL cell proliferation, MCL cell lines were treated with serial half-log dilutions of Compound 1 in 96-well format and assayed 72 hours later using a CellTiter-Glo® Assay (Promega) (FIG. 9A). The IC₅₀ values for Z-138, Jeko-1, Maver-1, Granta-519, and REC-1 cell lines are shown in FIG. 9B. These data show that Compound 1 demonstrated potent single-agent anti-proliferative activity in multiple MCL cell lines.

### Example 9

### Compound 1 as a Single Agent in MCL Xenograft Models

The effects of Compound 1 were assessed in two different mantle cell lymphoma (MCL) xenograft models. Jeko-1 and or Maver-1 cells were inoculated subcutaneously in the flanks of CB17.SCID (for Jeko-1 cells) or athymic nude (for Maver-1 cells) mice. Once the tumors reached a volume of about 200 mm³, the mice were randomized into study groups (n=10 per group). Mice were treated with Compound 1 by oral gavage, or with only vehicle as a negative control. In one treatment group, Compound 1 was administered once per day for 21 days. In the other treatment group, Compound 1 was administered twice per day for three cycles, wherein each cycle constituted treatment for three consecutive days, followed by no treatment for four consecutive days.

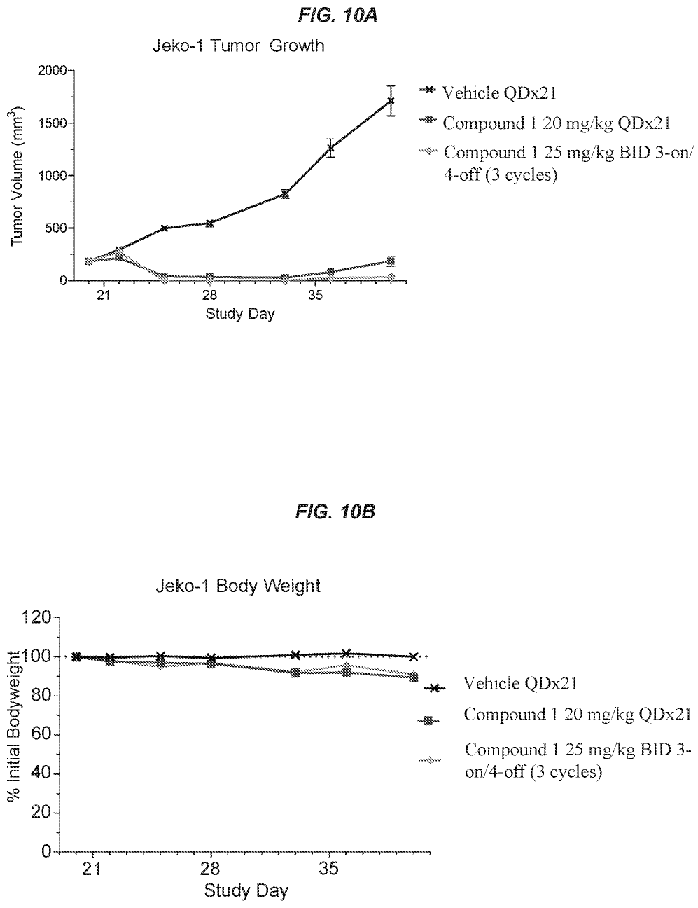

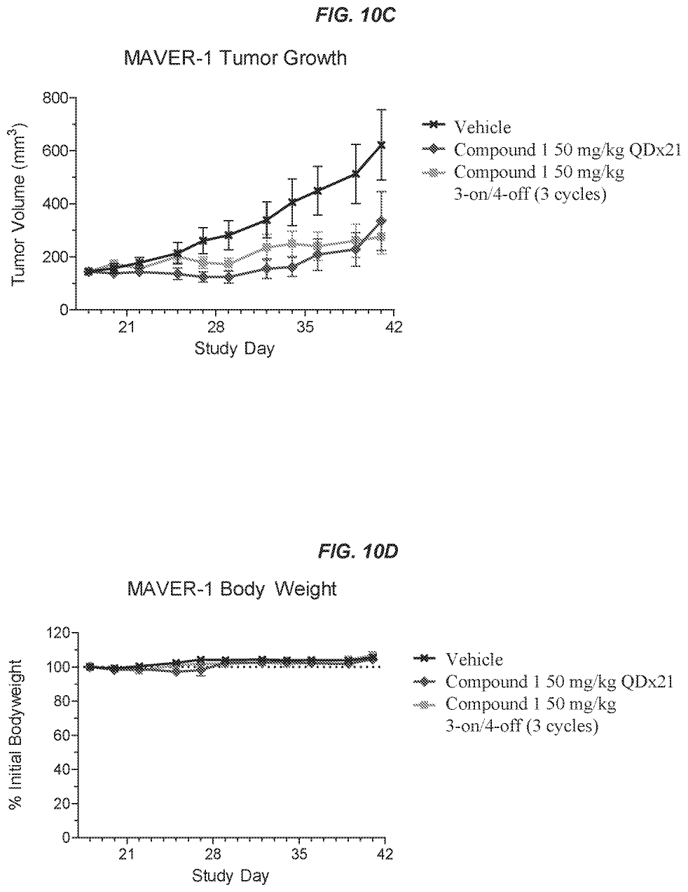

As shown in FIGS. 10A and 10C, both treatment regimens significantly inhibited tumor growth in both the Jeko-1 and Maver-1 models, respectively. Furthermore, FIGS. 10B and 10D shows that Compound 1 was well-tolerated.

### Example 10

### Synergism of Compound 1 and a Wee1 Inhibitor in MCL Cell Lines

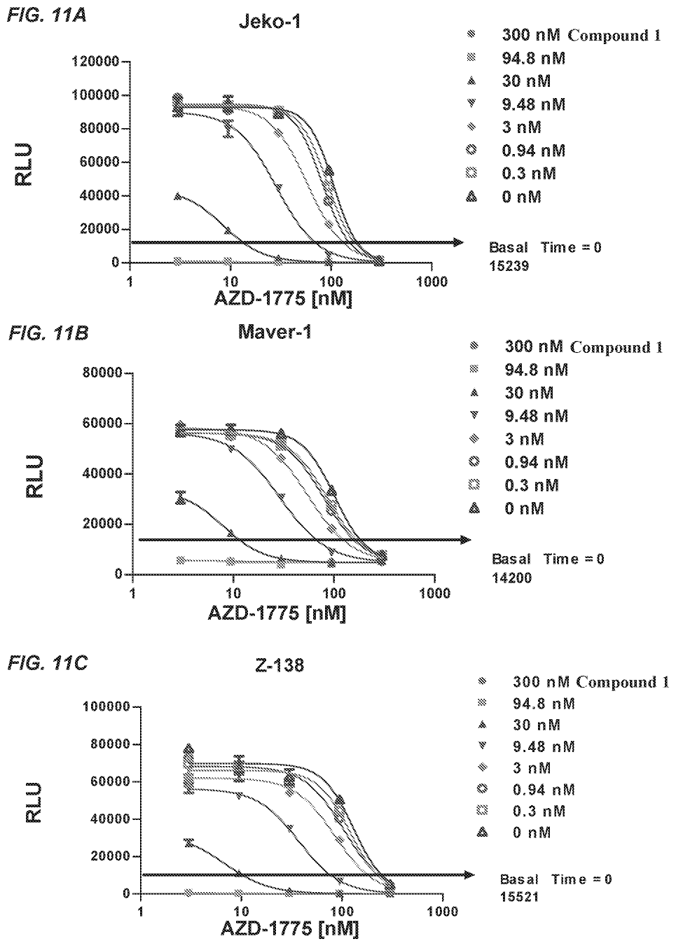

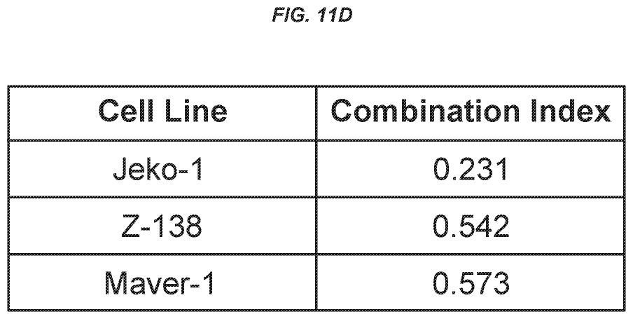

In vitro assays were performed in order to examine the effects of combining Compound 1 and a Wee1 inhibitor on several mantle cell lymphoma (MCL) cell lines. MCL cells were treated with a titration of the Wee1 inhibitor AZD-1775 in combination with increasing concentrations of Compound 1. Proliferation was measured at 72 hours. Inhibition was observed in assays of Jeko-1 (FIG. 11A), Maver-1 (FIG. 11B), and Z-138 (FIG. 11C) cell lines.

In addition, MCL cells were treated with equipotent ratios of Compound 1 and AZD-1775 and combination index (CI) values were calculated using the Chou-Talalay method and CalcuSyn software (Caner Res. 2020 Jan. 15; 70(2):440-6). The CI values for Jeko-1, Z-138, and Maver-1 cells are shown in FIG. 11D. These data show that Compound 1 in combination with a Wee1 inhibitor exhibited synergistic anti-proliferative effects in MCL cell lines.

### Example 11

### DNA Damage and Apoptosis Induction in MCL Cell Lines

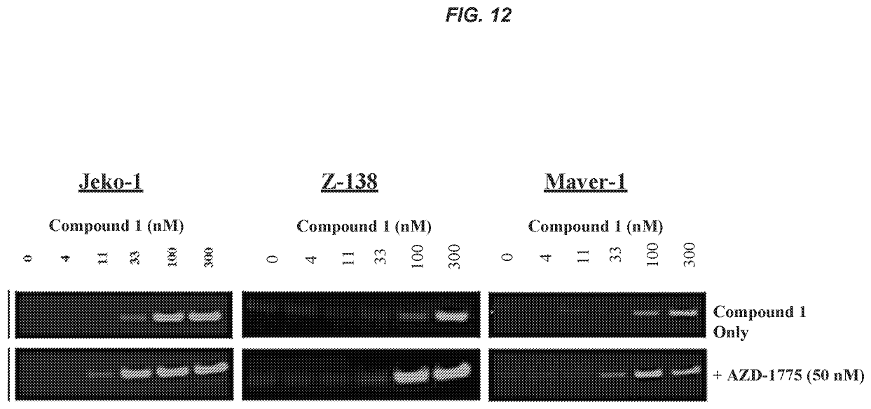

In order to assess the ability of Compound 1 to induce DNA damage, mantle cell lymphoma (MCL) cell lines were treated for 18 hours with a titration of Compound 1 alone or in combination with the Wee1 inhibitor AZD-1775. Cells were lysed and phospho-H2A.X (S139) levels were assayed by immunoblot and detected on a LI-COR Odyssey imager. As shown in FIG. 12, Compound 1 induced DNA damage in multiple MCL cell lines (Jeko-1, Z-138, and Maver-1 cells), and DNA damage induction was enhanced when Wee1 was concurrently inhibited.

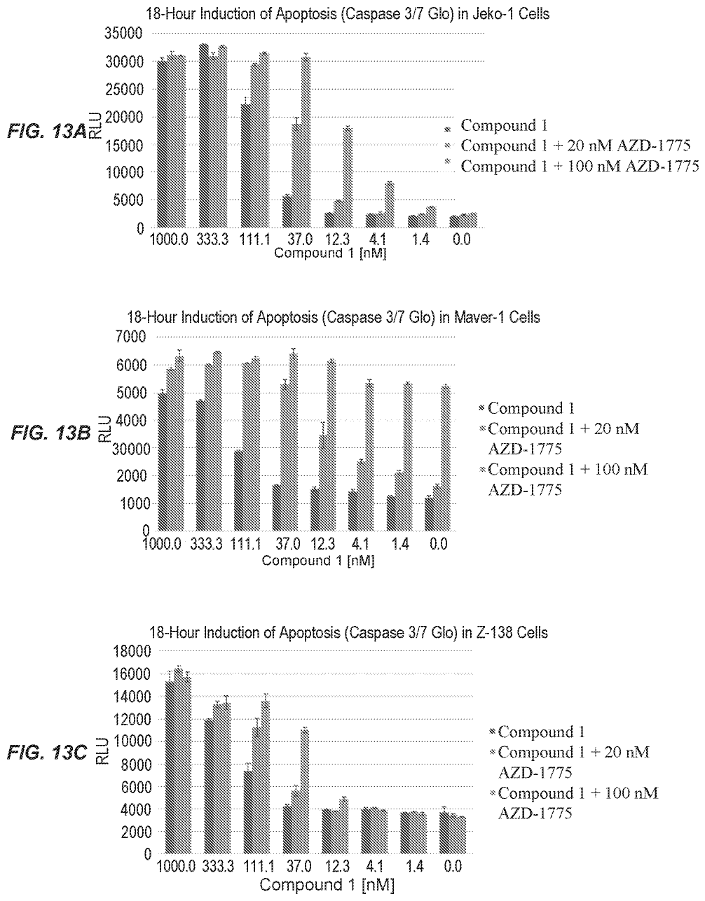

Apoptosis induction was studied by treating MCL cells for 18 hours with a titration of Compound 1 alone or in combination with AZD-1775. Following treatment, caspase-3/7 induction was assessed using a CaspaseGlo® 3/7 assay (Promega). The three MCL lines tested were Jeko-1 (FIG. 13A), Maver-1 (FIG. 13B), and Z-138 (FIG. 13C). These data show that the ability of Compound 1 to induce apoptosis in multiple MCL cell lines was enhanced by concurrent Wee1 inhibition.

### Example 12

### In Vivo Study of a Combination of Compound 1 and a Wee1 Inhibitor

A Jeko-1 mantle cell lymphoma (MCL) tumor xenograft model was used to study the combined effects of Chk1 inhibition with Compound 1 and a Wee1 inhibitor. Jeko-1 cells were inoculated subcutaneously into the flanks of CB17.SCID mice. Once tumors reached a volume of about 200 mm³, mice were randomized into study groups (n=10 per group). Mice were treated with Compound 1 (7.5 mg/kg), AZD-1775 (15 mg/kg), or both by oral gavage once per day for three cycles (each cycle consisting of three consecutive days of treatment followed by four consecutive days of no treatment). A negative control group was included wherein the mice were treated with vehicle only.

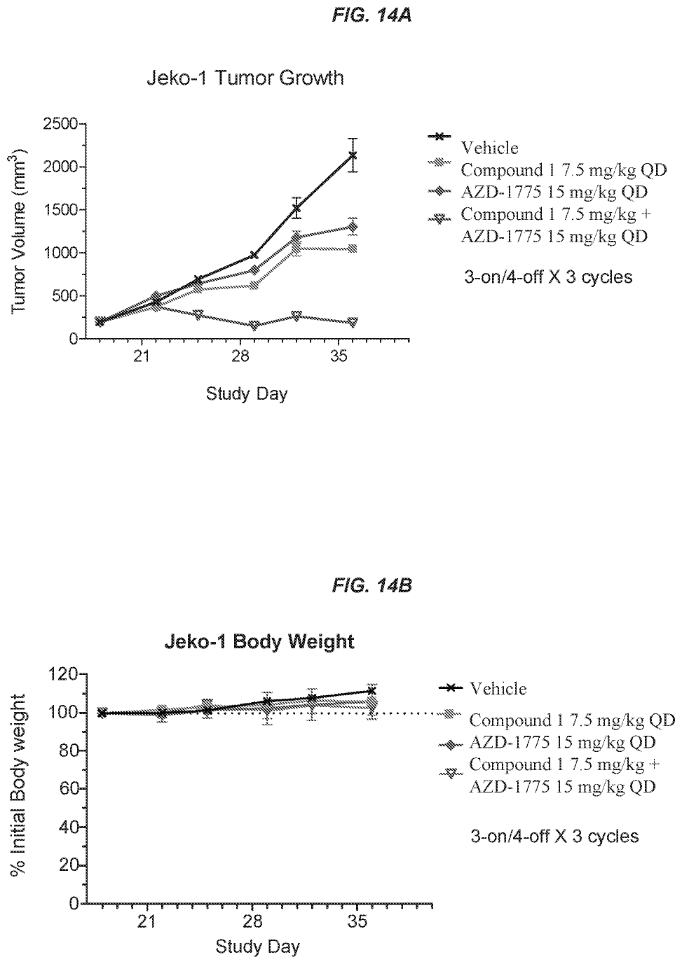

As shown in FIG. 14A, Jeko-1 tumor growth was inhibited by both agents alone, and inhibition of tumor growth was significantly enhanced when both agents were administered. FIG. 14B shows that Compound 1 and AZD-1775, both alone and in combination, were well-tolerated by the study animals. These results show that the anti-tumor activity of Compound 1 was enhanced with a Wee1 inhibitor in an MCL tumor model.

### Example 13

### In Vitro Study of Compound 1 Activity in AML Cell Lines

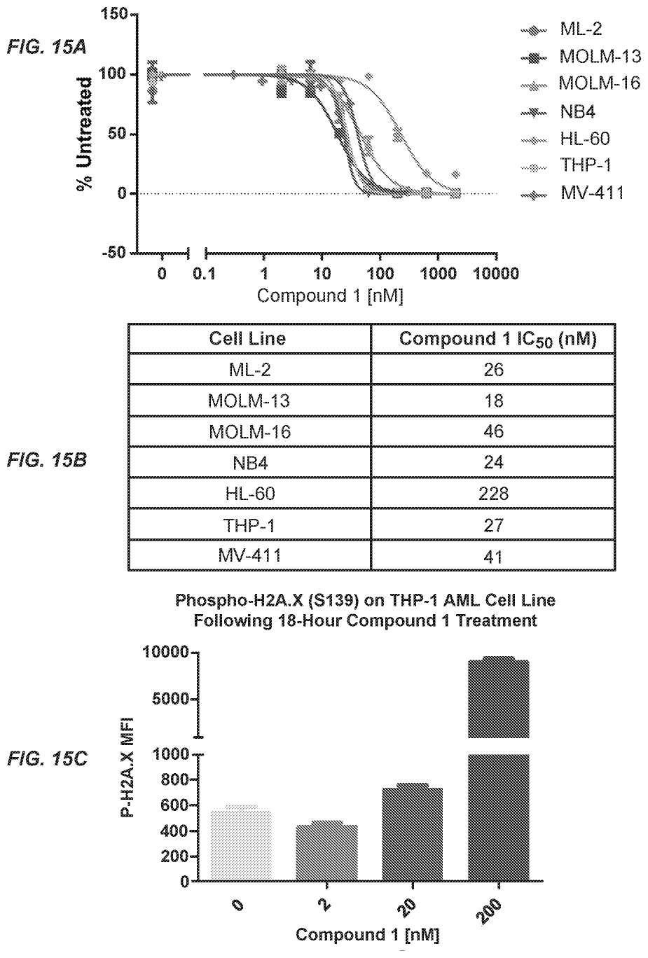

In order to examine the effects of Compound 1 on acute myeloid leukemia (AML) cells, several AML cell lines were treated with serial half-log dilutions of Compound 1 in a 96-well format and assayed for proliferation after 72 hours using a CellTiter-Glo® assay (Promega). As shown in FIGS. 15A and 15B, Compound 1 demonstrated anti-proliferative activity in multiple AML cell lines.

In addition, the ability of Compound 1 to induce DNA damage was assayed in THP-1 AML cells. For these experiments, THP-1 cells were treated with Compound 1 for 18 hours and phospho-H2A.X was measured using a Luminex assay (Millipore). As shown in FIG. 15C, Compound 1 was able to induce DNA damage in THP-1 cells in a dose-dependent manner.

### Example 14

### In Vivo Study of Compound 1 Activity in AML Cells

An MV-411 tumor xenograft model was used to test the ability of Compound 1 to inhibit AML tumor growth in vivo. MV-411 cells were mixed with Matrigel in a 1:1 ratio and injected subcutaneously into the right flanks of female NOD.SCID mice. Once tumors reached a volume of about 100 to 200 mm³, mice were randomized into study groups (n=10 per group) and dosed with Compound 1 by oral gavage.

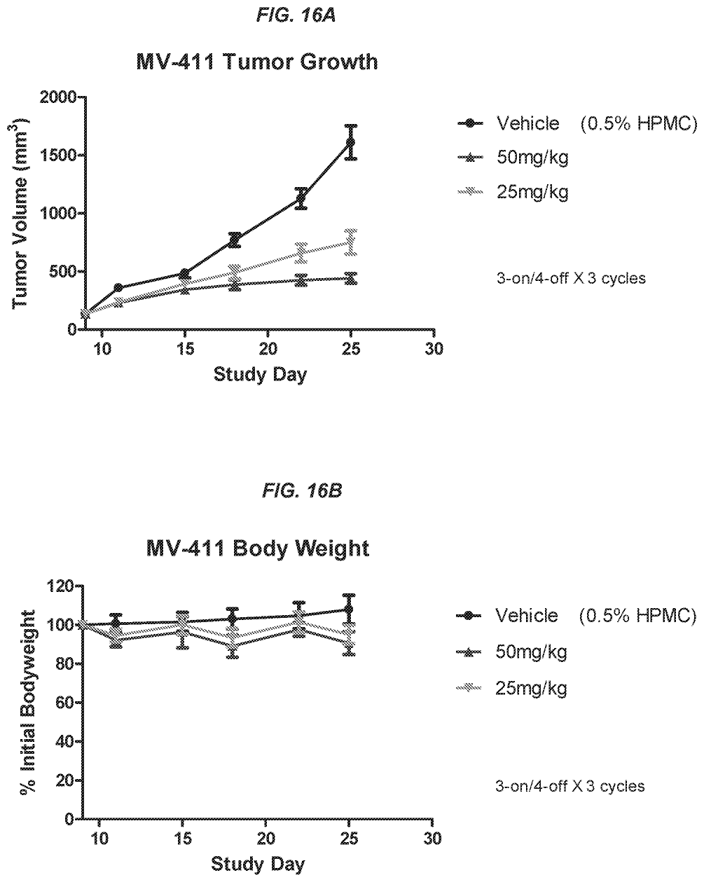

FIG. 16A shows that Compound 1 inhibited MV-411 tumor growth in a dose-dependent fashion. FIG. 16B shows the effects of Compound 1 on the body weight of the study animals. Together, these results show that Compound 1 was active and well-tolerated as a single agent in an MV-411 xenograft tumor model.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

### Informal Sequence Listing

| SEQ ID | | |
|---|---|---|
| NO: | Sequence | Description |
| 1 | KKKVSRSGLYRSPSMPEN | synthetic |
| | LNRPR | peptide |
| | | substrate |

What is claimed is:

1. A method for preventing or treating cancer in a subject, the method comprising administering to the subject a therapeutically effective amount of (i) Compound 1 or a pharmaceutically acceptable salt thereof; and (ii) a Wee1 inhibitor or a pharmaceutically acceptable salt thereof.

2. The method of claim 1, wherein the Wee1 inhibitor is AZD-1775.

3. The method of claim 1 or 2, wherein the cancer is selected from the group consisting of acute myeloid leukemia, esophageal cancer, gastric cancer, mantle cell lymphoma, non-small cell lung cancer (NSCLC), ovarian cancer, head and neck cancer, liver cancer, pancreatic cancer, prostate cancer, and a central nervous system cancer.

4. The method of any one of claims 1 to 3, wherein the cancer is a metastatic cancer.

5. The method of any one of claims 1 to 4, wherein the cancer is a multidrug-resistant cancer.

6. The method of any one of claims 1 to 5, wherein the dose of Compound 1 is between about 1 mg and 100 mg per kg of the subject's body weight.

7. The method of claim 6, wherein the dose of Compound 1 is about 12.5 mg per kg of the subject's body weight.

8. The method of claim 6, wherein the dose of Compound 1 is about 25 mg per kg of the subject's body weight.

9. The method of claim 6, wherein the dose of Compound 1 is about 50 mg per kg of the subject's body weight.

10. The method of any one of claims 2 to 9, wherein the dose of AZD-1775 is about 30 mg per kg of the subject's body weight.

11. The method of any one of claims 6 to 10, wherein the dose of Compound 1 is about 25 mg per kg of the subject's body weight and the dose of AZD-1775 is about 30 mg per kg of the subject's body weight.

12. The method of any one of claims 1 to 11, wherein Compound 1 and the Wee1 inhibitor are co-administered.

13. The method of claim 12, wherein Compound 1 and the Wee1 inhibitor are co-administered simultaneously or sequentially.

14. The method of any one of claims 1 to 13, wherein Compound 1 or the Wee1 inhibitor are administered orally, intravenously, intramuscularly, subcutaneously, or intratumorally.

15. The method of any one of claims 1 to 14, wherein treating the subject results in a reduction of tumor volume.

16. The method of any one of claims 1 to 15, wherein treating the subject results in a decrease or elimination of one or more signs or symptoms of cancer.

17. The method of any one of claims 1 to 16, wherein treating the subject results in an increased survival time.

18. The method of any one of claims 1 to 14, wherein the subject does not have cancer.

19. The method of any one of claims 1 to 14, wherein the method further comprises administering a DNA-damaging agent.

20. A pharmaceutical composition comprising:
(i) Compound 1 or a pharmaceutically acceptable salt thereof;
(ii) a Wee1 inhibitor or a pharmaceutically acceptable salt thereof; and
(iii) a pharmaceutically acceptable carrier.

21. The pharmaceutical composition of claim Error! Reference source not found 19, wherein the Wee1 inhibitor is AZD-1775.

22. The pharmaceutical composition of claim 20 or 21, wherein Compound 1 is present at a concentration between about 0.1 nM and 2,000 nM.

23. The pharmaceutical composition of claim 21 or 22, wherein AZD-1775 is present at a concentration between about 0.1 nM and 1,000 nM.

24. The pharmaceutical composition of any of claims 19 to 22, wherein the composition comprises a DNA-damaging agent.

25. A kit for preventing or treating cancer in a subject, the kit comprising a pharmaceutical composition of any one of claims 20 to 23.

26. The kit of claim 25, further comprising instructions for use.

27. The kit of claim 25 or 26, further comprising one or more reagents.

28. The kit of any of claims 25 to 27, wherein the composition comprises a DNA-damaging agent.


SEQUENCE LISTING (1 sequence)

SEQ ID NO: 1 - protein, 23 aa, Artificial Sequence
  Synthetic Construct
KKKVSRSGLYRSPSMPENLNRPR